SEQUENCE LISTING (15 sequences)

SEQ ID NO: 1 - DNA, 33 bp, Artificial Sequence
  primer_bind: primer VEGF Nterm
tggtcccaggctcatatggcacccatggcagaa

SEQ ID NO: 2 - DNA, 38 bp, Artificial Sequence
  primer_bind: primer VEGF Cterm
tctagaccggagccaccgccaccccgcctcggcttgtc

SEQ ID NO: 3 - DNA, 33 bp, Artificial Sequence
  primer_bind: primer Gel Nterm
ggtggcggtggctccggtctagacaccgtgagc

SEQ ID NO: 4 - DNA, 45 bp, Artificial Sequence
  primer_bind: primer Gel Cterm
aaggctcgtgtcgacctcgagtcattaagctttaggatctttatc

SEQ ID NO: 5 - DNA, 36 bp, Artificial Sequence
  primer_bind: primer NcoIgb
ggtggcggtggctccatggaaccaatcctgcttctg

SEQ ID NO: 6 - DNA, 39 bp, Artificial Sequence
  primer_bind: primer CxhoIgb
gccaccgcctccctcgagctattagtagcgtttcatggt

SEQ ID NO: 7 - protein, 5 aa, Artificial Sequence
  DOMAIN: a cleavage site for EK
DDDDK

SEQ ID NO: 8 - DNA, 39 bp, Artificial Sequence
  primer_bind: primer NgbEK
ggtaccgacgacgacgacaagatcatcgggggacatgag

SEQ ID NO: 9 - DNA, 30 bp, Artificial Sequence
  primer_bind: primer Cgb
ggagccaccgccaccgtagcgtttcatggt

SEQ ID NO: 10 - DNA, 30 bp, Artificial Sequence
  primer_bind: primer Nvegf
ggtggcggtggctccgcacccatggcagaa

SEQ ID NO: 11 - DNA, 42 bp, Artificial Sequence
  primer_bind: primer CxhoI veg
aaggctcgtgtcgacctcgagtcattaccgcctcggcttgtc

SEQ ID NO: 12 - DNA, 18 bp, Artificial Sequence
  primer_bind: primer T7 promoter
taatacgactcactatag

SEQ ID NO: 13 - DNA, 30 bp, Artificial Sequence
  primer_bind: primer CpET32EK
cttgtcgtcgtcgtcggtacccagatctgg

SEQ ID NO: 14 - DNA, 744 bp, Homo sapiens
  gene: human granzyme B with signal peptide sequence
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaa

SEQ ID NO: 15 - protein, 247 aa, Homo sapiens
  PROPEP: human granzyme B with signal peptide sequence
MQPILLLLAFLLLPRAGAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

(19) United States
(12) Patent Application Publication
(10) Pub. No.: US 2004/0248805 A1
(43) Pub. Date: Dec. 9, 2004

(54) Vascular endothelial growth factor fusion constructs and uses thereof

(76) Inventor: Michael Rosenblum, Sugarland, TX (US)

Correspondence Address: Dr. Benjamin Adler, ADLER & ASSOCIATES, 8011 Candle Lane, Houston, TX, 77071, US

(21) Appl. No.: 10/846,022

(22) Filed: May 14, 2004

Related U.S. Application Data
(60) Provisional application No. 60/476,209, filed on Jun. 5, 2003.

(51) Int. Cl. A61K 38/18; C07K 14/475

(52) U.S. Cl. 514/12; 530/399

(57) ABSTRACT

The 121-amino acid isoform of vascular endothelial growth factor (VEGF₁₂₁) is linked by a flexible G4S tether to a cytotoxic molecule such as toxin gelonin or granzyme B and expressed as a soluble fusion protein. The VEGF₁₂₁ fusion protein exhibits significant anti-tumor vascular-ablative effects that inhibit the growth of primary tumors and inhibit metastatic spread and vascularization of metastases. The VEGF₁₂₁ fusion protein may also target osteoclast precursor cells in vivo and inhibits osteoclastogenesis.

### CROSS-REFERENCE TO RELATED APPLICATION

This non-provisional patent application claims benefit of provisional patent application U.S. Ser. No. 60/476,209, filed Jun. 5, 2003, now abandoned.

### FEDERAL FUNDING LEGEND

This invention was produced in part using funds obtained through Grant 5P30CA16672-26 from the National Cancer Institute and National Institutes of Health Grant ROI CA 7495. Consequently, the federal government has certain rights in this invention.

### BACKGROUND OF THE INVENTION

1. Field of the Invention

The present invention relates generally to the fields of cancer research and targeted therapy. More specifically, the present invention relates to fusion constructs comprising the 121-amino acid isoform of vascular endothelial growth factor (VEGF₁₂₁) and uses of such contructs.

2. Description of the Related Art

Vascular endothelial growth factor (VEGF)-A plays a central role in the growth and metastasis of solid tumors, and acts as a primary stimulant of vascularization in solid tumors. VEGF-A enhances endothelial cell proliferation, migration, and survival and is essential for blood vessel formation. Other roles of vascular endothelial growth factor include wound healing, vascular permeability and the regulation of blood flow. Through alternative splicing of RNA, human vascular endothelial growth factor exists as at least four isoforms of 121, 165, 189, or 206 amino acids. The lowest molecular weight isoform, designated VEGF₁₂₁, is a non-heparan sulfate-binding isoform that exists in solution as a disulfide-linked homodimer.

VEGF is released by a variety of tumor cells. The angiogenic actions of VEGF are mediated through two related receptor tyrosine kinases, kinase domain receptor (KDR) and FLT-1 in the human, and Flk-1 and Flt-1 in the mouse. Both are largely restricted to vascular endothelial cells. KDR/Flk-1 and FLT-1 receptors are overexpressed on the endothelium of tumor vasculature. In contrast, these receptors are almost undetectable in the vascular endothelium of adjacent normal tissues. The receptors for vascular endothelial growth factor thus seem to be excellent targets for the development of therapeutic agents that inhibit tumor growth and metastatic spread through inhibition of tumor neovascularization.

To this end, VEGF₁₂₁ would be an appropriate carrier to deliver a toxic agent selectively to tumor vascular endothelium. VEGF₁₂₁ exists in solution as a disulfide linked homodimer and binds to KDR and FLT-1 in a heparin-independent manner. It does not bind neuropilin-1 or neuropilin-2. VEGF₁₂₁ has been shown to contain the full biological activity of the larger variants.

Molecular engineering enabled the synthesis of novel chimeric molecules having therapeutic potential. Chimeric fusion constructs targeting the IL-2 receptor, the EGF receptor, and other growth factor/cytokine receptors have been described. It has also been showed that a chemical conjugate of vascular endothelial growth factor and truncated diphtheria toxin has impressive cytotoxic activity on cell lines expressing receptors for vascular endothelial growth factor. Further studies with VEGF/diphtheria toxin fusion constructs demonstrated selective toxicity to Caprice's sarcoma cells and dividing endothelial cells in vitro and in vivo. However, the prior art is deficient in fusion constructs comprising vascular endothelial growth factor and other cytotoxic molecule with improved biochemical and pharmacological properties. The present invention fulfills this long-standing need and desire in the art.

### SUMMARY OF THE INVENTION

The present invention discloses targeting of neovasculature of solid tumors with a chimeric fusion toxin comprising the 121-amino acid isoform of vascular endothelial growth factor (VEGF₁₂₁). In one embodiment, the chimeric fusion toxin (VEGF₁₂₁/rGel) consists of VEGF₁₂₁ and recombinant gelonin (rGel), a low molecular weight single chain toxin with a mechanism of action similar to that of ricin A-chain. VEGF₁₂₁ is linked by a flexible G4S tether to the toxin gelonin and expressed as a soluble protein in bacteria. Both VEGF₁₂₁/rGel and VEGF₁₂₁ stimulated cellular kinase domain receptor (KDR) phosphorylation. The VEGF₁₂₁/rGel fusion construct was highly cytotoxic to endothelial cells overexpressing the KDR/Flk-1 receptor. Endothelial cells overexpressing FLT-1 were not sensitive to the fusion protein.

While several studies have shown both receptors of VEGF₁₂₁, namely VEGFR-1 (FLT-1) and VEGFR-2 (KDR/Flk-1), to be over-expressed on the endothelium of tumor vasculature, the present invention reports several surprising results which demonstrate that VEGF₁₂₁/rGel has several advantageous properties. Cell ELISA using antibodies specific to either KDR or FLT-1 indicate binding of VEGF₁₂₁/rGel to both receptors. While VEGF₁₂₁/rGel binds to both FLT-1 and KDR, internalization of VEGF₁₂₁/rGel is mediated only by KDR and not FLT-1.

Experiments with human melanoma (A-375) or human prostate (PC-3) xenografts demonstrate successful use of VEGF₁₂₁/rGel fusion construct for the targeted destruction of tumor vasculature in vivo. The present invention also indicates that the anti-tumor vascular-ablative effect of VEGF₁₂₁/rGel may be utilized not only for treating primary tumors but also for inhibiting metastatic spread and vascularization of metastases. Taken together, these results indicate that selective destruction of tumor vasculature can be achieved with VEGF₁₂₁/rGel in mice, giving impressive antitumor effects. Gross morphological toxicity to normal organs was not visible in animals treated with a therapeutic dose. Therefore, VEGF₁₂₁/rGel is a potential antitumor agent useful for treating cancer patients.

In another embodiment of the present invention, there is provided a chimeric fusion toxin (GrB/VEGF₁₂₁) consists of VEGF₁₂₁ and granzyme B (GrB), a serine protease capable of inducing apoptosis through both caspase-dependent and caspase-independent pathways. GrB/VEGF₁₂₁ induced apoptotic events specifically on FLK-1-expressing porcine aortic endothelial cells as assessed by terminal deoxynucleotidyl transferase-mediated nick end labeling assay, DNA laddering, and cytochrome c release from mitochondria. In addition, the fusion construct mediated cleavage of caspase-8, caspase-3, and poly(ADP-ribose) polymerase in target endothelial cells within 4 h after treatment. In conclusion, delivery of the human proapoptotic pathway enzyme granzyme B to tumor vascular endothelial cells or to tumor cells may have significant therapeutic potential and represents a potent new class of targeted therapeutic agents with a unique mechanism of action.

The present invention is directed to a composition of matter comprising a conjugate comprising the 121-amino acid isoform of vascular endothelial growth factor (VEGF₁₂₁) and a cytotoxic molecule. In general, the cytotoxic molecule is a toxin such as gelonin or a molecule that induces apoptosis such as granzyme B.

In another embodiment of the present invention, there is provided a method of using the VEGF₁₂₁ fusion conjugate of the present invention to kill cells expressing type 2 VEGF receptors (kinase domain receptor/Flk-1 receptors). The VEGF₁₂₁ component of the conjugate binds to both VEGF receptor type 1 (Flt-1) and VEGF receptor type 2 (KDR/Flk-1) but is only internalized by cells expressing VEGF receptor type 2.

In yet another embodiment of the present invention, there is provided a method of using the VEGF₁₂₁ fusion conjugate of the present invention to inhibit tumor growth or inhibit metastatic spread and vascularization of metastases in an animal or a human.

The present invention further provides a method of using the VEGF₁₂₁ fusion conjugate of the present invention to inhibit osteoclastogenesis or treat osteoporosis in an animal or a human.

Other aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows the design and construction of VEGF₁₂₁/rGel. Constructs of the targeting molecule (VEGF₁₂₁) to the cytotoxic agent (gelonin) were expressed in two orientations, with either VEGF₁₂₁ or gelonin at the N-terminus. A G4S tether was used to fuse VEGF₁₂₁ and gelonin and reduce steric hindrance.

FIG. 2 shows a rabbit reticulocyte assay used to determine the ability of VEGF₁₂₁/rGel and rGel to inhibit translation in a cell-free system. The fusion of VEGF₁₂₁ and recombinant gelonin does not reduce the activity of the toxin component.

FIG. 3 shows an ELISA demonstrating that VEGF₁₂₁/rGel binds to the receptor. VEGF₁₂₁/rGel, VEGF₁₂₁ and rGel were incubated with biotinylated mouse flk-1 receptor attached to NeutrAvidin-coated plates. Binding was assessed using anti-gelonin and anti-VEGF antibodies.

FIG. 4 shows binding to flk-1 receptor is specific for VEGF₁₂₁/rGel. VEGF₁₂₁/rGel or VEGF₁₂₁ was incubated with flk-1 receptor. Binding of VEGF₁₂₁/rGel was competed with VEGF₁₂₁ and a rabbit anti-gelonin antibody was used for detection. VEGF₁₂₁ specifically reduced binding of VEGF₁₂₁/rGel to flk-1. VEGF₁₂₁ was not detected by the anti-gelonin antibody (data not shown).

FIG. 5 shows cytotoxicity of VEGF₁₂₁/rGel to KDR-expressing porcine aortic endothelial cells (PAE). Cells transfected with either the FLT-1 or KDR receptor were treated with various doses of VEGF₁₂₁/rGel or rGel for 72 h. Cells expressing the FLT-1 receptor were equally insensitive to VEGF₁₂₁/rGel and rGel (IC₅₀/300 nM). In contrast, cells expressing KDR were about 200-fold more sensitive to the fusion construct (IC₅₀ of 0.5 nM) than they were to rGel.

FIGS. 6A-B show expression of KDR and FLT-1. FIG. 6A: Whole cell lysate (30 μg) of PAE/KDR and PAE/FLT-1 was run on an SDS-PAGE gel, transferred to a PVDF membrane and immunoblotted using the appropriate antibody. Expression of both receptors on their respective cell lines was confirmed. FIG. 6B: Receptor-specific binding of radio-labeled VEGF₁₂₁/rGel is demonstrated on cells expressing these receptors. Binding was reduced with unlabeled VEGF₁₂₁/rGel but not by unlabeled gelonin.

FIG. 7 shows internalization of VEGF₁₂₁/rGel into PAE/KDR and PAE/FLT-1 cells. PAE/KDR cells were incubated with 4 μg/ml VEGF₁₂₁/rGel at the timepoints indicated. Cells were then incubated with an anti-gelonin polyclonal antibody (1:200) followed by a FITC-conjugated secondary antibody (1:80). Nuclei were stained with propidium iodide. VEGF₁₂₁/rGel enters PAE/KDR cells within one hour of treatment. However, PAE/FLT-1 cells did not internalize VEGF₁₂₁/rGel even after 24 hours of incubation with VEGF₁₂₁/rGel.

FIG. 8 shows the effect of exposure time of VEGF₁₂₁/rGel on PAE/KDR cells on cytotoxicity. VEGF₁₂₁/rGel was incubated with PAE/KDR cells for varying lengths of time. While VEGF₁₂₁/rGel retained cytotoxicity towards PAE/KDR cells even with a 1 h exposure time, cytotoxicity of this fusion toxin was markedly enhanced by an exposure time of 48 hours.

FIG. 9 shows that cytotoxicity of VEGF₁₂₁/rGel to PAE/KDR cells does not result in apoptosis. PAE/KDR cells were grown overnight. 1 nM VEGF₁₂₁/rGel (twice the IC₅₀) was added and incubated for 24, 48 and 72 hours. The cells were analyzed for TUNEL. Positive control cells were incubated with 1 mg/ml DNAse for 10 minutes at 37° C.

FIG. 10 shows that treatment of PAE/KDR cells with VEGF₁₂₁/rGel does not result in PARP cleavage. PAE/KDR cells were stimulated with VEGF₁₂₁/rGel or VEGF₁₂₁ for the times indicated. Cells were washed and lysed and the cell lysate was analyzed by Western using an anti-PARP antibody. No PARP cleavage was observed.

FIG. 11 shows inhibition of human melanoma growth in mice by VEGF/rGel. Groups of nude mice bearing A-375M tumors were treated intravenously with saline, rGel, or fusion construct every 2-3 days for 11 days. Administration of rGel did not affect tumor growth. Treatment with VEGF₁₂₁/rGel at a total dose of either 17 mg/kg or 25 mg/kg significantly suppressed tumor growth. However, treatment at the 25 mg/kg dose level resulted in mortality by day 19. None of the animals dosed at 17 mg/kg showed gross evidence of toxicity.

FIG. 12 shows inhibition of human prostate carcinoma growth in mice by VEGF/rGel. Groups of nude mice bearing PC-3 tumors were treated intravenously with saline, rGel, or the VEGF₁₂₁/rGel fusion construct (20 mg/kg total dose) every 2-3 days for 11 days. Administration of rGel (10 mg/kg) had no effect on tumor growth. In contrast, treatment with the fusion construct completely inhibited tumor growth for 26 days and resulted in a 7-fold reduction in tumor volume compared with saline-treated or rGel-treated controls.

FIG. 13 shows the specific localization of VEGF/rGel to tumor vasculature in PC3 tumors. Nude mice bearing human prostate PC-3 tumors were injected i.v. with VEGF₁₂₁/rGel or rGel (2.5 mg/kg). Thirty minutes after administration, tissues were removed and snap frozen. Sections were stained with immunofluorescent reagents to detect murine blood vessels (MECA-32, red) and with antirGel (green). Vessels stained with both reagents appear yellow. VEGF/rGel localized to tumor vessels, whereas rGel did not. Vessels in all normal organs other than the kidney (glomerulus) were unstained by VEGF/rGel.

FIG. 14 shows the destruction and thrombosis of tumor blood vessels by VEGF/rGel. Nude mice bearing human prostate PC-3 tumors were treated i.v. with one dose of VEGF₁₂₁/rGel (2.5 mg/kg). Forty-eight hours after administration, tissues were snap-frozen, sectioned, and stained with hematoxylin and eosin. As shown in this representative image, tumors from mice treated with the fusion construct had damaged vascular endothelium. Clots were visible in the larger vessels of the tumors, and erythrocytes were visible in the tumor interstitium, indicating a loss of vascular integrity. In contrast, histological damage was not visible in any normal organs, including the kidneys, of treated mice.

FIGS. 15A-B show VEGF₁₂₁/rGel is not cytotoxic to MDA-MB-231 cells. Log-phase MDA-MB-231 cells were treated with various doses of VEGF₁₂₁/rGel or rGel for 72 hrs. The cytotoxic effects of both agents were similar, indicating no specific cytotoxicity of the fusion construct compared to free toxin on these cells (FIG. 15B). Western analysis demonstrated the presence of VEGFR-2 on endothelial cells transfected with the R2 receptor (PAE/KDR) but not on cells expressing the FLT-1 receptor (PAE/FLT-1, negative control). The MDA-MB-231 cells did not express detectable amounts of VEGFR-2 (FIG. 15A).

FIG. 16 shows VEGF₁₂₁/rGel localizes to blood vessels of MDA-MB-231 tumor. Mice bearing orthotopically-placed MDA-MB-31 tumors were administered one dose (i.v., tail vein) of VEGF₁₂₁/rGel. Four hours later, the mice were sacrificed and tumors excised and fixed. Tissue sections were stained for blood vessels using the Meca 32 antibody (red) and the section was counter-stained using an anti-gelonin antibody (green). Co-localization of the stains (yellow) demonstrate the presence of the VEGF₁₂₁/rGel fusion construct specifically in blood vessels and not on tumor cells.

FIG. 17 shows VEGF₁₂₁/rGel reduces number of large metastatic colonies in lungs. The size of tumor colonies was analyzed on slides stained with 6 w/32 antibody that specifically recognizes human HLA antigens. The antibody delineates colonies of human tumor cells and defines borders between metastatic lesions and mouse lung parenchyma. The largest size differences between VEGF₁₂₁/rGel and control groups were found in groups of colonies having diameter either less than 50 μm or more than 1000 μm. In the VEGF₁₂₁/rGel-treated mice more than 40% of total foci were extremely small (<50 micron) as compared to 18% in the control group. The control mice had approximately 8% of extremely large colonies (>1000 μm) whereas VEGF₁₂₁/rGel-treated mice did not have colonies of this size.

FIGS. 18A-B show VEGF₁₂₁/rGel inhibits vascularization of MDA-MB-231 pulmonary metastases. Lungs derived from VEGF₁₂₁/rGel and rGel—treated mice were stained with MECA 32 antibody and the number of vessels per mm² within the metastatic foci was determined (FIG. 18A). The mean number of vessels per mm² in lung metastases of VEGF₁₂₁/rGel-treated mice was reduced by approximately 50% as compared to those in rGel-treated mice. FIG. 18B shows representative images demonstrating reduction of vascular density in foci of comparable size in mice treated with rGel (left) and VEGF₁₂₁/rGel fusion protein (right).

FIG. 19 shows VEGF₁₂₁/rGel inhibits proliferation of metastatic MDA-MB-23 1 cells in the lungs. Frozen sections of lungs derived from VEGF₁₂₁/rGel or rGel-treated mice were stained with Ki-67 antibody. Stained sections were examined under x40 objective to determine the number of tumor cells with positive nuclei (cycling cells). Positive cells were enumerated in 10 colonies per slide on six sections derived from individual mice per each treatment group. The mean number per group ±SEM is presented. VEGF₁₂₁/rGel treatment reduced the average number of cycling cells within the metastatic foci by approximately 60%.

FIG. 20 shows detection of VEGFR-2 on vasculature of metastatic lesions by anti-VEGFR-2 antibody RAFL-1. Frozen sections of lungs from mice treated with VEGF₁₂₁/rGel or free gelonin were stained with monoclonal rat anti-mouse VEGFR-2 antibody RAFL-1 (10 μg/ml). RAFL-1 antibody was detected by goat anti-rat IgG-HRP. Sections were developed with DAB and counterstained with hematoxylin. Representative images of lung metastases of comparable size (700-800 μm in the largest diameter) from each treatment group are shown. Images were taken with an objective of X20. Note that the pulmonary metastases from the VEGF₁₂₁/rGel treated group show both reduced vessel density and decreased intensity of anti-VEGFR-2 staining compared to control lesions.

FIG. 21 shows VEGF₁₂₁/rGel strongly inhibits the growth of prostate cancer cells PC-3 placed in the bone micro-environment in mice. Animals were anesthesized prior to injection of 50,000 PC-3 cells into the distal epipysis of the right femur. Treatment with VEGF₁₂₁/rGel or saline (control) began one week after tumor placement. The maximum tolerated dose of VEGF₁₂₁/rGel was utilized and administered i.v. as shown. Tumor growth was monitored by X-ray and animals with large osteolytic lesions or bone lysis were sacrificed. All control mice were sacrificed by day 67. In contrast, 50% of the VEGF₁₂₁/rGel-treated mice survived past day 140 without sign of osteolysis.

FIG. 22 shows the effects of VEGF₁₂₁/rGel and rGel on RANKL-mediated osteoclast formation. Raw 264.7 cells were cultured overnight in 24-well plates.

Osteoclast formation was induced by addition of 100 ng/ml RANKL with increasing concentrations of VEGF₁₂₁/rGel or rGel. Cells were allowed to differentiate for 96 hours followed by determination of the number of osteoclasts per well. Each experiment was performed in triplicate. The data shown is representative of three separate experiments. RANKL or RANKL+rGel-treated Raw 264.7 cells differentiate into large multi-nucleated TRAP-positive osteoclasts. In contrast, RANKL+VEGF₁₂₁/rGel-treated cells do not differentiate and do not stain for TRAP.

FIG. 23 shows cloning of human granzyme B (GrB) gene from HuT-78 cells. HuT-78 RNA was isolated, and premature GrB cDNA (˜800 bp) was amplified by reverse transcription-PCR and cloned into the PCR 2.1 TA vector. The human granzyme B sequence with 20-amino acid signal sequence was confirmed and designated as premature granzyme B. Once the signal peptide was removed, the mature amino-terminal Ile-Ile-Gly-Gly sequence of granzyme B was generated.

FIG. 24 shows the construction of GrB/VEGF₁₂₁ fusion toxin by PCR and insertion into the pET32a(+) vector. Mature granzyme B was attached to the recombinant VEGF₁₂₁ carrier via a flexible tether (G4S). A cleavage site for EK (DDDDK) was inserted upstream and adjacent to the first amino acid isoleucine of granzyme B. The fused gene fragment was then introduced into XbaI and XhoI sites of the pET32a(+) vector to form the expression vector pET32GrB/VEGF₁₂₁.

FIGS. 25A-B show bacterial expression, purification, and Western blot analysis of the GrB/VEGF₁₂₁ fusion toxin. FIG. 25A: 8.5% SDS-PAGE and Coomassie blue staining under reducing conditions showed that GrB/VEGF₁₂₁ was expressed as a 55-kDa molecule with tags and the size of the final purified GrB/VEGF₁₂₁ was ˜38 kDa. FIG. 25B: Western blotting confirmed that the fusion protein reacted with either mouse anti-VEGF or mouse anti-GrB antibody.

FIGS. 26A-B show GrB/VEGF₁₂₁ bound to PAE/FLK-1 cells but not to PAE/FLT-1 cells, A375 M or SKBR3 cells. Binding of GrB/VEGF₁₂₁ to cells was assessed by 96-well ELISA plates coated with 50,000 cells/well of PAE/FLK-1, PAE/FLT-1, A375 M or SKBR3 cells. The wells were blocked with 5% BSA and then treated with purified GrB/VEGF₁₂₁ at various concentrations. The wells were then incubated with either anti-GrB antibody (FIG. 26A) or anti-VEGF antibody (FIG. 26B) followed by HRP-goat anti-mouse IgG. ABTS solution with 1 ml/ml of 30% H₂O₂ were added to the wells, and absorbance at 405 nm was measured after 30 min.

FIG. 27 shows internalization of GrB/VEGF₁₂₁ into porcine aortic endothelial (PAE) cells. PAE cells were plated onto 16-well chamber slides (1×10⁴ cells/well), treated with 100 nM of GrB/VEGF₁₂₁ for 4 h and then washed briefly with PBS. The cell surface was stripped with glycine buffer (pH 2.5) and the cells were fixed in 3.7% formaldehyde and permeabilized in PBS containing 0.2% Triton X-100. After blocking, samples were incubated with anti-granzyme B antibody and treated with FITC-coupled anti-mouse IgG. The slides were analyzed under a fluorescence microscope. The granzyme B moiety of GrB/VEGF₁₂₁ was delivered into the cytosol of PAE/FLK-1 but not into that of PAE/FLT-1 cells after 4-h treatment.

FIG. 28A shows cytotoxicity of the GrB/VEGF₁₂₁ fusion toxin on transfected endothelial cells. Log-phase PAE cells were plated into 96-well plates at a density of 2.5×10³ cells/well and allowed to attach for 24 h. The medium was replaced with medium containing different concentrations of GrB/VEGF₁₂₁. After 72 h, the effect of fusiontoxin on the growth of cells in culture was determined using XTT. Plates were read on a microplate ELISA reader at 540 nm. IC₅₀ of GrB/VEGF₁₂₁ was ˜10 nM on PAE/FLK-1 cells; it was not cytotoxic on PAE/FLT-1 cells.

FIG. 28B shows growth inhibitory effects of GrB/VEGF₁₂₁ as determined by colony-forming assay. PAE cells (5×10⁵ cells/ml) were incubated at 37° C. and 5% CO₂ for 72 h with different concentrations of GrB/VEGF₁₂₁ and 100 nM of irrelevant fusion protein GrB/scFvMEL. Cells were then washed with PBS, trypsinized, counted, and diluted serially. The serial cell suspensions were then plated in triplicate and cultured in six-well plates for 5-7 days. Cells were stained with crystal violet and colonies consisting of >20 cells were counted. The results are shown as percentage of colonies in relation to the number of colonies formed by untreated cells.

FIGS. 29A-B show GrB/VEGF₁₂₁ induces apoptosis on PAE/FLK-1 cells. Cells (1×10⁴ cells/well) were treated with GrB/VEGF₁₂₁ at an IC₅₀ concentration for different times (0, 24, and 48 h) and washed with PBS. Cells were fixed with 3.7% formaldehyde and permeabilized with 0.1% Triton X-100 and 0.1% sodium citrate. Cells were incubated with TUNEL reaction mixture, incubated with Converter-AP, and finally treated with Fast Red substrate solution. The slides were analyzed under a light microscope. Apoptosis cells were stained red (400×) (FIG. 29A). FIG. 29B shows apoptotic cells as percentage of the total counted cells (>200 cells) in randomly selected fields (200×); bars, SD.

FIG. 30 shows granzyme B/VEGF₁₂₁ induces cytochrome c release from mitochondria to cytosol and Bax translocation from cytosol to mitochondria. PAE cells (5×10⁷) were treated with granzyme B/VEGF₁₂₁ at concentrations of 0, 0.1, and 20 nM for 24 h. Cells were collected, and the cytosolic and mitochondrial fractions were isolated as described below. Fractions of 30 mg each from non-treated and treated cells were loaded onto 15% SDS-PAGE gels, and standard Western blotting procedure was performed. The blot was probed with anti-cytochrome c antibody or anti-Bax antibody.

FIG. 31 shows GrB/VEGF₁₂₁ induces DNA laddering in PAE/FLK-1 cells. Cells were plated into six-well plates at a density of 2×10⁵ cells/well and exposed to 20 nM GrB/VEGF₁₂₁ for 24 h. DNA was isolated from cell lysates and fractionated on 1.5% agarose gel.

FIG. 32 shows cleavage and activation of caspase-3, caspase-8, and PARP in PAE/FLK-1 cells treated with GrB/VEGF₁₂₁. PAE cells were plated into six-well plates at a density of 2×10⁵ cells/well and treated with 20 nM GrB/VEGF₁₂₁ for 4 h. The total cell lysates were loaded onto 12% SDS-PAGE and Western blot was performed using appropriate primary antibodies.

### DETAILED DESCRIPTION OF THE INVENTION

The expression of vascular endothelial growth factor and its receptors has been closely linked to tumor vascularity, metastasis, and progression. Several groups have developed anti-angiogenic drugs that block kinase activity of the vascular endothelial growth factor receptors or monoclonal antibodies that block vascular endothelial growth factor-receptor interactions. The present invention demonstrates chimeric fusion constructs containing the 121-amino acid isoform of vascular endothelial growth factor (VEGF₁₂₁) and a cytotoxic molecule such as plant toxin gelonin or serine protease granzyme B.

Agents targeting the neovascularization process in tumors have significant potential for therapeutic impact. Molecules which interfere with the growth and development of vascular endothelial cells by targeting the VEGF/receptor complex have an additional advantage since these agents do not have to penetrate into the tumor parenchyma and the receptor targets are expressed on the luminal surface of tumor vascular endothelium.

Possible binding of vascular endothelial growth factor-containing constructs to the neuropilin receptor could be a source of unwanted toxicity and mis-targeting of the complex; however, it has been shown that the VEGF₁₂₁ fragment as opposed to other isoforms of VEGF-A does not appear to bind to this receptor.

It is specifically contemplated that pharmaceutical compositions may be prepared using the novel fusion constructs of the present invention. In such a case, the pharmaceutical composition comprises the novel fusion constructof the present invention and a pharmaceutically acceptable carrier. A person having ordinary skill in this art would readily be able to determine, without undue experimentation, the appropriate dosages and routes of administration of this fusion toxin of the present invention. When used in vivo for therapy, the fusion construct of the present invention is administered to the patient or an animal in therapeutically effective amounts, i.e., amounts that eliminate or reduce the tumor burden or other desired biological effects. It will normally be administered parenterally, preferably intravenously, but other routes of administration will be used as appropriate.

The dose and dosage regimen will depend upon the nature of the disease or cancer (primary or metastatic) and its population, the characteristics of the particular fusion toxin, e.g., its therapeutic index, the patient, the patient's history and other factors. The amount of fusion toxin administered will typically be in the range of about 0.01 to about 100 mg/kg of patient weight. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics, 8th Ed. (1990) Pergamon Press. For parenteral administration, the fusion toxin protein will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used.

Liposomes may be used as carriers. The vehicle may contain minor amounts of additives such as substances that enhance isotonicity and chemical stability, e.g., buffers and preservatives. The fusion toxin will typically be formulated in such vehicles at concentrations of about 0.01 mg/ml to 1000 mg/ml.

As used herein, a “subject” refers to an animal or a human.

The present invention is directed to a composition of matter comprising a conjugate comprising the 121-amino acid isoform of vascular endothelial growth factor (VEGF₁₂₁) and a cytotoxic molecule. In general, the cytotoxic molecule is a toxin such as gelonin or a signal transduction protein capable of generating apoptotic signals. Representative useful signal transduction proteins include granzyme B and Bax. In one embodiment, the conjugate is a fusion protein in which VEGF₁₂₁ and the cytotoxic molecule are linked by a linker such as G₄S, (G₄S)2, the 218 linker, (G₄S)3, enzymatically cleavable linker or pH cleavable linker or any similar such linker as would be well known to a person having ordinary skill in this art.

In another embodiment of the present invention, there is provided a method of using the VEGF₁₂₁ fusion conjugate of the present invention to kill cells expressing type 2 VEGF receptors (kinase domain receptor/Flk-1 receptors). The VEGF₁₂₁ component of the conjugate binds to both VEGF receptor type 1 (Flt-1) and VEGF receptor type 2 (KDR/Flk-1) but is only internalized by cells expressing VEGF receptor type 2. In general, the conjugate is cytotoxic to cells expressing more than 2000 type 2 VEGF receptors per cell.

In yet another embodiment of the present invention, there is provided a method of using the VEGF₁₂₁ fusion conjugate of the present invention to inhibit tumor growth or inhibit metastatic spread and vascularization of metastases in an animal or a human. The method involves the use of a biologically effective amount of the conjugate to exert cytotoxic effect on the tumor vasculature. The method may further comprise treatment with chemotherapeutic agents or radiotherapeutic agents. Representative chemotherapeutic and radiotherapeutic agents are well-known in the art.

The present invention further provides a method of using the VEGF₁₂₁ fusion conjugate of the present invention to inhibit osteoclastogenesis or treat osteoporosis in an animal or a human.

The following examples are given for the purpose of illustrating various embodiments of the invention and are not meant to limit the present invention. The present examples, along with the methods, procedures, treatments, and specific compounds described herein are representative of preferred embodiments. One skilled in the art will appreciate that the present invention is well adapted to carry out the objects and obtain the advantages mentioned, as well as those objects, ends and advantages inherent herein. Changes therein and other uses which are encompassed within the spirit of the invention as defined by the scope of the claims will occur to those skilled in the art.

### EXAMPLE 1

Cell Lines and Reagents

Endothelial cell growth supplement from bovine neural tissue was obtained from Sigma. Murine brain endothelioma bEnd.3 cells were provided by Werner Risau (Max Plank Institute, Munich, Germany). Porcine aortic endothelial cells (PAE) transfected with either the human FLT-1 receptor (PAE/FLT-1) or the KDR receptor (PAE/KDR) were provided by Dr. J. Waltenberger. Soluble mouse Flk-1 was expressed in Sf9 cells as described by Warren et al. (1995). The human melanoma A-375 M cell line, human breast cancer SKBR3-HP, and HuT-78 cells were obtained from American Type Culture Collection. Tissue culture reagents were from GIBCO/BRL or Mediatech Cellgro (Herndon, Va.).

Rabbit anti-gelonin antisera was obtained from the Veterinary Medicine Core Facility at M.D. Anderson Cancer Center. Anti-flt-1 (sc-316), anti-flk-1 (sc-504), and anti-PARP (sc-8007) antibodies were purchased from Santa Cruz Biotechnology, Inc. (Santa Cruz, Calif.). BALB/c nude mice were purchased from The Jackson Laboratory and maintained under sterile pathogen-free conditions according to American Association of Laboratory Animal Care standards.

Anti-granzyme B mouse monoclonal antibody, and anti-caspase antibodies were obtained from Santa Cruz Biotechnology (Santa Cruz, Calif.). Horseradish peroxidase-goat anti-mouse (HRP-GAM) or anti-rabbit conjugate were purchased from Bio-Rad (Hercules, Calif.). FITC-coupled anti-mouse IgG was obtained from Sigma Chemical Co. (St. Louis, Mo.). Cytochrome c release apoptosis assay kit was purchased from Oncogene Research Products (Boston, Mass.). In situ cell death detection kit, AP [terminal deoxynucleotidyl transferase-mediated nick end labeling (TUNEL) assay], and Fast Red were from Roche Molecular Biochemicals (Indianapolis, Ind.).

The PCR reagents were obtained from Fisher Scientific, and the molecular biology enzymes were purchased from Roche Molecular Biochemicals or New England Biolabs. Bacterial strains, pET bacterial expression plasmids, and recombinant enterokinase were obtained from Novagen. All other chemicals were obtained from Sigma or Fisher Scientific. Metal affinity resin (Talon) was obtained from CLONTECH. Other chromatography resin and materials were purchased from Amersham Pharmacia.

### EXAMPLE 2

Construction of VEGF₁₂₁/rGelonin Fusion Toxin

The cDNA encoding human VEGF₁₂₁ and recombinant gelonin were fused together by using the splice overlap extension PCR method with VEGF and gelonin DNA as templates. Primers used were: VEGF Nterm, (5′-TGGTCCCAGGCTCATATGGCACCCATGGCAGAA-3′, SEQ ID NO. 1); VEGF Cterm, (5′-TCTAGACCGGAGCCACCGCCACCCCGCCTCGGCTTGTC-3′, SEQ ID NO. 2); Gel Nterm, (5′-GGTGGCGGTGG CTCCGGTCTAGACACCGTGAGC-3′, SEQ ID NO. 3); Gel Cterm, (5′-AAG GCTCGTGTCGACCTCGAGTCATTAAGCTTTAGGATCTTTATC-3′, SEQ ID NO. 4). A G4S linker was incorporated between the VEGF₁₂₁ and the rGel sequences. Purified PCR products were digested with the restriction enzymes BspHI and XhoI and ligated into pET-32a. The constructs were transformed into Escherichia coli strain AD494 (DE3) pLys S for expression of the fusion protein.

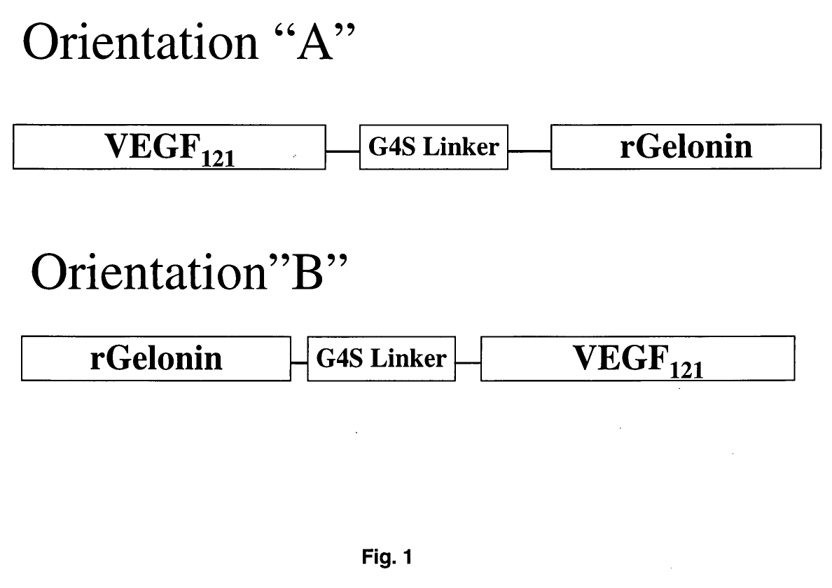

The combination of VEGF₁₂₁ and recombinant gelonin was originally prepared in two different orientations (FIG. 1) with both orientations displaying similar cytotoxicity profiles. However, the orientation with VEGF₁₂₁ at the N-terminus results in a higher yield following purification from bacteria, and is used in subsequent experiments.

### EXAMPLE 3

VEGF₁₂₁/rGelonin Expression in E. coli and Purification

The expression and purification of VEGF₁₂₁/rGel has been previously described (Veenendaal et al., 2002). Bacterial colonies transformed with the plasmid carrying the VEGF₁₂₁/rGel insert were cultured in LB growth medium (Sigma) containing 200 mg/ml ampicillin, 70 mg/ml chloramphenicol, and 15 mg/ml kanamycin at 37° C. overnight in a shaker bath at 240 rpm. The cultures then were diluted 1:20 with fresh LB medium with antibiotics and grown to early log phase (A600/0.6) at 37° C. Thereafter, the cultures were diluted 1:1 with fresh LB medium plus antibiotics. Protein synthesis was induced at 23° C. by the addition of 0.1 mM isopropyl b-D-thiogalactoside (IPTG) overnight. The cells were collected by centrifugation, resuspended in 10 mM Tris/HCl (pH 8.0), and frozen.

The fusion protein was expressed and purified from bacterial supernatant. E. coli cells were lysed with 100 ml 0.1 mm glass beads (BioSpec Products, Inc) in a Bead Beater (BioSped Products, Inc) for eight cycles of 3 minutes each. The lysate was ultracentrifuged at 40,000 rpm for 90 minutes at 4° C. The supernatant was carefully collected and adjusted to 40 mM Tris-HCl (pH 8.0), 300 mM NaCl, and incubated at 4° C. with metal affinity resin. The resin was washed with 40 mM Tris-HCl (pH8.0), 0.5 M NaCl buffer containing 5 mM Imidazole and eluted with buffer containing 100 mM Imidazole. After pooling fractions containing VEGF₁₂₁/rGel, the sample was dialyzed against 20 mM Tris (pH 8.0), 200 mM NaCl and digested with recombinant Enterokinase at room temperature. Enterokinase was removed by agarose-linked soybean trypsin inhibitor. Other proteins of non-interest were removed by Q Sepharose Fast Flow resin and metal affinity resin as described previously²⁶. VEGF₁₂₁/rGel was concentrated and stored in sterile PBS at −20° C.

SDS/PAGE analysis of protein expression after induction with IPTG showed a new protein at 62 kDa, which is the expected molecular weight for the fusion protein plus the 21 kDa purification tag. This material was purified by binding and elution from IMAC resin. Cleavage with recombinant enterokinase removed the tag resulting in a 42-kDa protein under reducing conditions. The construct migrated as a homodimer at 84 kDa under nonreducing conditions. The fusion construct was immunoreactive with antibodies to both VEGF and rGel. One liter of induced bacterial culture initially contained ˜2,000 mg of soluble fusion construct. Initial IMAC purification resulted in 750 mg of VEGF₁₂₁/rGel product (yield 37.5%), and digestion with recombinant enterokinase generated 400 mg of target protein (yield 20%). Subsequent purification yielded 230 mg of VEGF₁₂₁/rGel final product (11.5% overall yield).

### EXAMPLE 4

Anti-VEGF and Anti-rGel Western Blot Analysis

Protein samples were analyzed by SDS/15% PAGE under reducing conditions. The gel was electrophoretically transferred to nitrocellulose overnight at 4° C. in transfer buffer (25 mM Tris/HCl, pH 7.6/190 mM glycine/20% HPLC-grade methanol). The membranes were blocked by the addition of 5% BSA in Western blocking buffer [(TBS)/Tween] and then incubated for 1 h with rabbit anti-gelonin polyclonal antibody (2 mg/ml in TBS/Tween) or mouse anti-VEGF monoclonal antibody 2C3 (2 mg/ml in TBS/Tween). The membrane then was incubated with goat-anti-rabbit IgG horseradish peroxidase (HRP) or goat-anti-mouse IgG-HRP (1:5,000 dilution in TBS/Tween). Then, the membrane was developed with the Amersham Pharmacia enhanced chemiluminescence (ECL) detection system and exposed to x-ray film.

### EXAMPLE 5

Biological Activity of the rGel Component

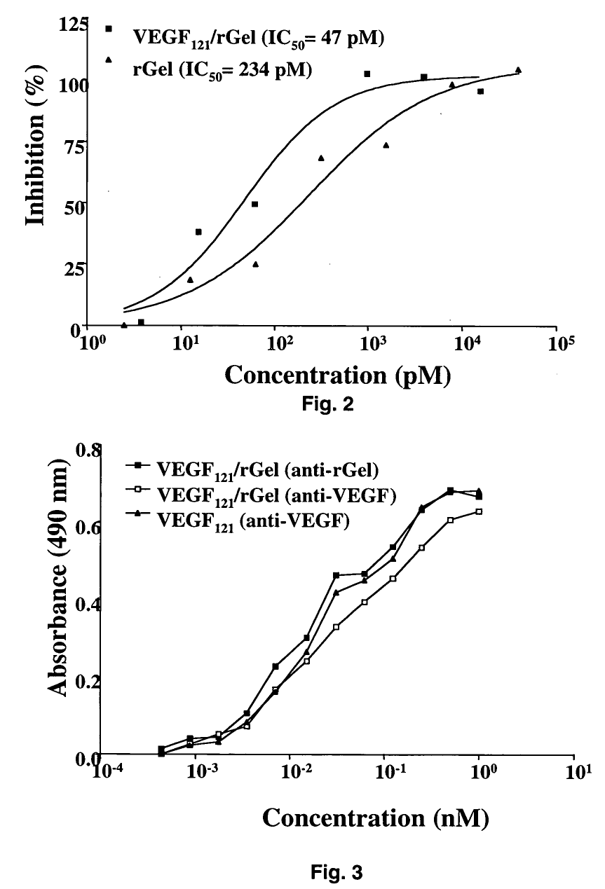

The functional activity of rGel and VEGF₁₂₁/rGel were assayed by using a cell-free protein translation inhibition assay kit from Amersham Pharmacia as described by the manufacturer. As determined by the rabbit reticulocyte translation assay, the purified VEGF₁₂₁/rGel and rGel had IC₅₀ values of ˜47 and 234 pM, respectively, showing that fusion of rGel and VEGF₁₂₁ did not reduce the activity of the toxin component (FIG. 2).

### EXAMPLE 6

Binding of VEGF₁₂₁/rGel to Soluble Flk-1 Receptor

Binding to Flk-1 was tested on microtiter plates coated with soluble mouse Flk-1. Plates were treated with 2 mg/ml of NeutrAvidin (Pierce) for 6 h. Purified, biotinylated Flk-1 (Warren et al., 1995) was incubated with NeutrAvidin-coated wells for 2 h. VEGF₁₂₁ or VEGF₁₂₁/rGel was added to the wells at various concentrations in the presence of PBS containing 2% (vol/vol) BSA. After 2 h of incubation, plates were washed and incubated with nonblocking mouse monoclonal anti-VEGF antibody, 2C3 (Brekken et al., 1998), or rabbit polyclonal anti-gelonin IgG. For competition studies of VEGF₁₂₁/rGel and VEGF₁₂₁, binding of the VEGF₁₂₁/rGel fusion protein was detected by using a rabbit anti-gelonin antibody. Mouse and rabbit IgG were detected by HRP-labeled goat anti-mouse and anti-rabbit antibodies, respectively (Dako). Peroxidase activity was measured by adding O-phenylenediamine (0.5 mg/ml) and hydrogen peroxide (0.03% vol/vol) in citrate-phosphate buffer (pH 5.5). The reaction was stopped by the addition of 100 ml of 0.18 M of H₂SO₄. The absorbance was read at 490 nM. In competition experiments, a 10-fold molar excess of VEGF₁₂₁ was premixed with VEGF₁₂₁/rGel before addition to the plate.

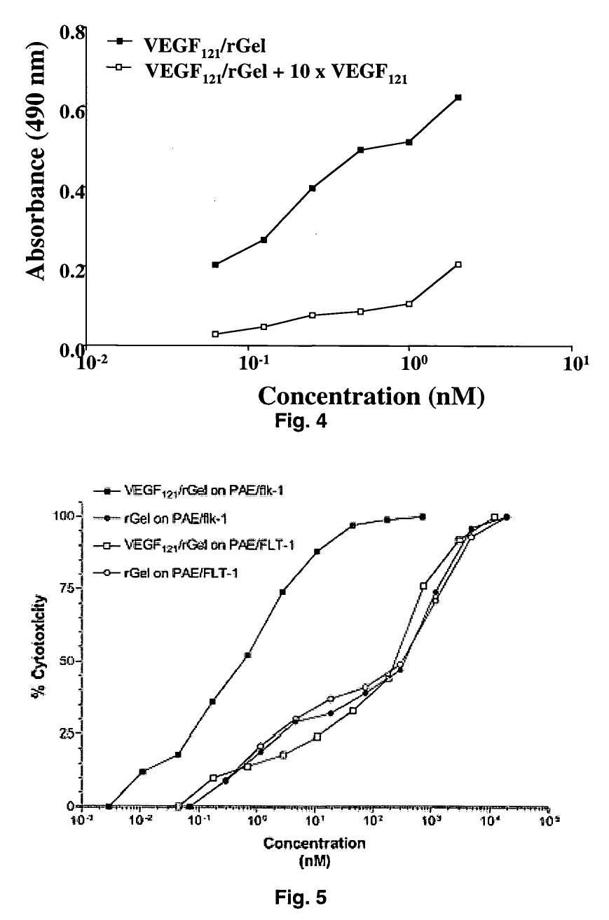

As shown in FIG. 3, VEGF₁₂₁/rGel and native human VEGF₁₂₁ bind equally well to Flk-1 at all concentrations, indicating that the VEGF component of the fusion protein is fully capable of binding to Flk-1. The specificity of binding of VEGF₁₂₁/rGel to Flk-1 was confirmed by using a 10-fold molar excess of free VEGF₁₂₁ (FIG. 4).

### EXAMPLE 7

VEGF₁₂₁/rGel and VEGF₁₂₁-Induced Phosphorylation of KDR

Porcine aortic endothelial cells (PAE/KDR) overexpressing the kinase domain receptor (KDR) were incubated overnight in F-12 culture medium and then incubated at 37° C. for 5 min with 100 mM Na3 VO4. VEGF or VEGF₁₂₁/rGel then were added and, at various times, cells were lysed by the addition of a lysis buffer (50 mM Hepes, pH 7.4/150 mM NaCl/1 mM EGTA/10 mM sodium pyrophosphate/1.5 mM MgCl2/100 mM NaF/10% (vol/vol) glycerol/1% Triton X-100). Cell lysates were centrifuged (16,000×g), the supernatants were removed, and their protein concentrations were determined. Lysate supernatants were incubated with 9 mg anti-phosphotyrosine monoclonal antibody (Santa Cruz Biotechnology) for 2 h at 4° C. and then precipitated by the addition of Protein A Sepharose beads for 2 h at 4° C. Beads were washed and mixed with SDS sample buffer, heated for 5 min at 100° C., centrifuged, analyzed by SDS/10% PAGE, and then transferred to nitrocellulose filters. The membranes were blocked with 5% nonfat dry milk and incubated with rabbit polyclonal anti-KDR antibody (1:250; Santa Cruz Biotechnology) for 1 h at room temperature. The membranes then were washed, incubated with a peroxidase-linked goat anti-rabbit antibody (1:2,000) for 1 h at room temperature, and then enhanced chemiluminescence reagent (Amersham Pharmacia) was used to visualize the immunoreactive bands.

Results from these experiments showed that addition of VEGF₁₂₁/rGel or VEGF₁₂₁ increased phosphotyrosine content. There were two phases of phosphorylation; an early phase (1-10 min) and a later phase (4-8 h). The time course of induction of KDR phosphorylation was the same for VEGF₁₂₁/rGel and VEGF₁₂₁. Phosphorylation of FLT-1 in PAE/FLT-1 cells treated with either VEGF₁₂₁/rGel or VEGF₁₂₁ was not observed, as expected from the weaker signaling of FLT-1 compared with KDR observed by others.

Although VEGF₁₂₁/rGel induces phosphorylation of KDR receptor, no growth-stimulatory effects of the fusion toxin on VEGF receptor-expressing cells were observed. These findings are in keeping with studies of other fusion toxins such as IL-2/DT that initially stimulate target cells in a manner similar to that of IL-2 itself, but ultimately kill the target cells through the actions of the internalized toxin.

### EXAMPLE 8

Cytotoxicity of VEGF₁₂₁/rGel to Endothelial Cells in vitro

To determine cytotoxicity on adult bovine aortic arch-derived endothelial cells (ABAE), log-phase adult bovine aortic arch-derived endothelial cells in DMEM [10% (vol/vol) FBS] were diluted to 4,000 cells per 200 ml. Aliquots (200 ml) were added to 96-well flat-bottomed tissue culture plates and incubated at 37° C. for 1-72 h in 5% CO₂. Purified VEGF₁₂₁/rGel or rGel were diluted in culture medium to various concentrations, added to the plate, and the cultures were incubated for 72 h. Remaining adherent cells were stained by the addition of 100 ml of crystal violet [0.5% in 20% (vol/vol) methanol]. Dye-stained cells were solubilized by the addition of 100 ml of Sorenson's buffer [0.1M sodium citrate, pH 4.2 in 50% (vol/vol) ethanol]. The absorbance was measured at 595 nM.

To determine cytotoxicity on mouse brain-derived endothelial cells bEnd.3, the cells were seeded at a density of 50,000/well in 24-well plates. Twenty-four hours later, VEGF₁₂₁/rGel or rGel alone were added at various concentrations. After 5 days of treatment at 37° C., remaining attached cells were trypsinized and counted. The results are presented as total cell number per well. Two identical experiments were performed in duplicate. Standard error in all experiments was less than 5% of the mean.

To determine cytotoxicity on PAE/KDR and PAE/FLT-1 cells, log-phase PAE/KDR cells and PAE/FLT-1 cells in F-12 medium [10% (vol/vol) FBS] were diluted to 3,000 cells per 200 ml. Aliquots (200 ml) were added to 96-well flat-bottomed tissue culture plates and incubated at 37° C. for 24 h in 5% CO₂. Purified VEGF₁₂₁/rGel or rGel were diluted in culture medium, added to the plate, and incubated for 72 h. Adherent cells were quantified by using the crystal violet staining method described above.

VEGF₁₂₁/rGel was specifically toxic to KDR/Flk-1 expressing endothelial cells in vitro (FIG. 5 and Table 1). The IC₅₀ values for VEGF₁₂₁/rGel on log-phase PAE/KDR, ABAE, and bEnd.3 cells, which express 1-3×10⁵ KDR/Flk-1 receptors per cell, was 0.06 to 1 nM. Cells expressing FLT-1 and having low endogenous expression of KDR (PAE/FLT-1, HUVEC) were several hundred-fold more resistant to VEGF₁₂₁/rGel than were the KDR/Flk-1 expressing cells. Thus, FLT-1 appears not to mediate cytotoxicity of VEGF₁₂₁/rGel.

The ratio of IC₅₀ values of rGel to VEGF₁₂₁/rGel was calculated for each cell type. This ratio (the targeting index) represents the ability of the VEGF component of the fusion construct to mediate the delivery of the toxin to the endothelial cell surface and into the intracellular ribosomal compartment. As summarized in Table 1, bEnd.3 and adult bovine aortic arch-derived endothelial cells were, respectively, 100-fold and 9-fold more sensitive to the fusion construct than they were to free rGel.

TABLE 1
Number of VEGF Receptors Per Cell And Sensitivity To VEGF₁₂₁/rGel

| | Number of | Number of | IC₅₀ for | | |
|---|---|---|---|---|---|
| | FLT-1 sites | KDR sites | VEGF₁₂₁/rGel | IC₅₀ for | Targeting |
| Cell Type | per cell | per cell | (nM) | rGel (nM) | index* |
| PAE/KDR | 0 | 2-3 × 10⁵ | 0.5 | 300 | 600 |
| (log phase) | | | | | |
| PAE/KDR | 0 | 2-3 × 10⁵ | 30 | 5000 | 167 |
| (confluent) | | | | | |
| bEnd3 (log phase) | Not done | 2 × 10⁵ | 1 | 100 | 100 |
| ABAE (log phase) | 0 | 0.4 × 10⁵ | 0.059 | 0.524 | 8.9 |
| HUVEC | Not done | 0.023 × 10⁵ | 700 | >1000 | ˜1 |
| (hypoxia) | | | | | |
| HUVEC | Not done | 0.017 × 10⁵ | 800 | >1000 | ˜1 |
| (normoxia) | | | | | |
| PAE/FLT-1 | 0.5 × 10⁵ | Not done | 300 | 300 | 1 |
| (log phase) | | | | | |
| PAE/FLT-1 | 0.5 × 10⁵ | Not done | >5000 | 10000 | <2 |
| (confluent) | | | | | |
| A-375 (log phase) | Not done | Not done | 330 | 109 | 0.3 |
| PC-3 (log phase) | Not done | Not done | 225 | 100 | 0.4 |

*Targeting index is defined as the ratio of IC₅₀ of rGel to VEGF₁₂₁/rGel.

### EXAMPLE 9

Selective Cytotoxicity of VEGF₁₂₁/rGel for Dividing PAE/KDR Cells

VEGF₁₂₁/rGel was 60-fold more toxic to PAE/KDR cells in log-phase growth than it was to PAE/KDR cells that had been grown to confluence and rested (Table 1). This effect was not caused by differences in KDR expression, because the cells expressed the same number of KDR receptors per cell in both phases of growth. The log-phase PAE/KDR cells also were more sensitive to rGel itself than were the confluent cells, suggesting that the quiescence of confluent cells impacts their sensitivity to both targeted and nontargeted rGel. It is possible that the rate or route of entry of both VEGF₁₂₁/rGel and rGel is different for dividing and nondividing cells.

### EXAMPLE 10

VEGF₁₂₁/rGel Binds to Both KDR and FLT-1

VEGF₁₂₁ has been shown to bind to the FLT-1 receptor with greater affinity than to KDR. Because cytotoxicity of VEGF₁₂₁/rGel to KDR-expressing cells was found to be nearly 600-fold greating than for FLT-1 expressing cells, the relative binding of VEGF₁₂₁/rGel to PAE cells expressing each of the receptors was investigated.

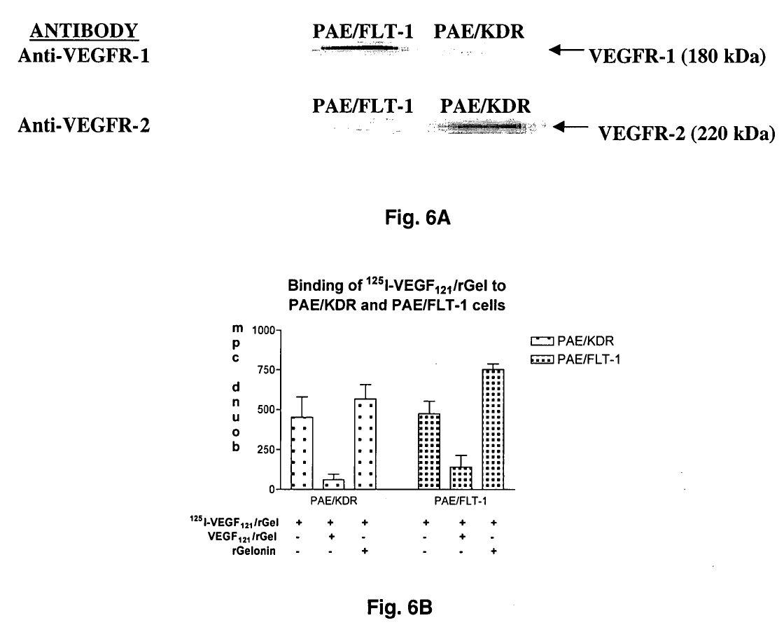

ELISA analysis was performed to confirm the expression of both receptors on the cell surface using receptor-specific antibodies (data not shown). Expression of VEGFR-1 (FLT-1) and VEGFR-2 (KDR) was confirmed by western blot (FIG. 6A). Whole cell lysates of PAE/KDR and PAE/FLT-1 cells were obtained by lysing cells in Cell Lysis buffer (50 mM Tris, pH 8.0, 0.1 mM EDTA, 1 mM DTT, 12.5 mM MgCl₂, 0.1 M KCl, 20% glycerol) supplemented with protease inhibitors (0.5% leupeptin, 0.5% aprotinin and 0.1% PMSF). Protein samples were separated by SDS-PAGE under reducing conditions and electrophoretically transferred to a PVDF memberane overnight at 4^{B}C in transfer buffer (25 mM Tris-HCl, pH 7.6, 190 mM glycine, 20% HPLC-grade methanol). The samples were analyzed for KDR with rabbit anti-flk-1 polyclonal antibody and FLT-1 using an anti-flt-1 polyclonal antibody. The membranes were then incubated with goat-anti-rabbit IgG horseradish peroxidase (HRP), developed using the Amersham ECL detection system and exposed to X-ray film.

In order to confirm that VEGF₁₂₁/rGel bound to human VEGFR-1 and VEGFR-2 and that the presence of recombinant gelonin did not interfere with the binding properties of VEGF₁₂₁, the binding of radiolabeled VEGF₁₂₁/rGel to both KDR and FLT-1 receptors expressed on the surface of PAE cells was investigated. One hundred μg of VEGF₁₂₁/rGel was radiolabeled with 1 mCi of NaI¹²⁵ using Chloramine T²⁷ for a specific activity of 602 Ci/mMol. Cells were grown overnight in 24-well plates. Non-specific binding sites were blocked for 30 minutes with PBS/0.2% gelatin followed by incubation for 4 hours with ¹²⁵I-VEGF₁₂₁/rGel in PBS/0.2% gelatin solution. For competition experiments, cold VEGF₁₂₁/rGel or gelonin were pre-mixed with ¹²⁵I-VEGF₁₂₁/rGel. Cells were washed four times with PBS/0.2% gelatin solution, detached and bound cpm was measured.

FIG. 6B shows that the binding of ¹²⁵I-VEGF₁₂₁/rGel to both cells was nearly identical. Binding of VEGF₁₂₁/rGel to both PAE/KDR and PAE/FLT-1 cells was competed by unlabeled VEGF₁₂₁/rGel but not by unlabeled gelonin, indicating that binding of VEGF₁₂₁/rGel was mediated by VEGF₁₂₁.

### EXAMPLE 11

Internalization of VEGF₁₂₁/rGel into PAE/KDR Cells

The internalization of VEGF₁₂₁/rGel into PAE/KDR and PAE/FLT-1 cells was investigated using immunofluorescence staining. PAE/KDR and PAE/FLT-1 cells were incubated with 4 μg/ml VEGF₁₂₁/rGel at various time points. After stripping the cell surface, cells were fixed with 3.7% formaldehyde and permeabilized with 0.2% Triton X-100. Non-specific binding sites were blocked with 5% BSA in PBS. Cells were then incubated with a rabbit anti-gelonin polyclonal antibody (1:200) followed by a TRITC-conjugated anti-rabbit secondary antibody (1:80). Nuclei were stained with propidium iodide (1 μg/ml) in PBS. The slides were fixed with DABCO media, mounted and visualized under fluorescence (Nikon Eclipse TS1000) and confocal (Zeiss LSM 510) microscopes.

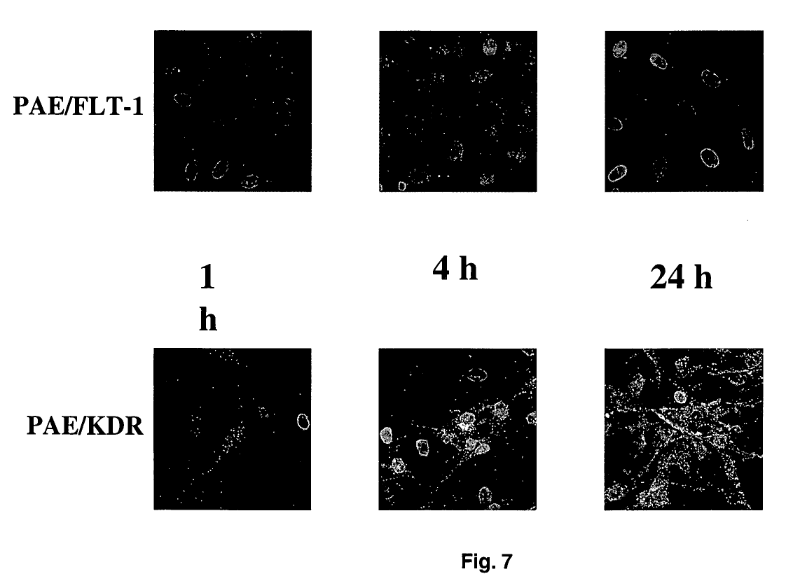

VEGF₁₂₁/rGel was detected in PAE/KDR cells within 1 hour of treatment with the immunofluoresence signal progressively increasing to 24 hours (FIG. 7). As expected, cell density also decreased over the 24 hour time period. No VEGF₁₂₁/rGel was detected in PAE/FLT-1 cells up to 24 hours after treatment with the fusion toxin. Treatment of cells with the same concentration of rGelonin showed no internalization, confirming that entry of VEGF₁₂₁/rGel into PAE cells was specifically via the KDR receptor.

### EXAMPLE 12

Cytotoxic Effects of VEGF₁₂₁/rGel as a Function of Exposure Time on Endothelial Cells

The IC₅₀ of VEGF₁₂₁/rGel incubated for 72 hours on log-phase PAE/KDR cells has been shown to be about 1 nM. However, VEGF₁₂₁/rGel internalizes into these cells within one hour of incubation. To study the cytototoxic effect of VEGF₁₂₁/rGel as a function of exposure time of this agent on endothelial cells, cells were incubated with VEGF₁₂₁/rGel from 1-72 hours and its cytotoxicity on PAE/KDR cells was assessed at the end of the 72-hour period.

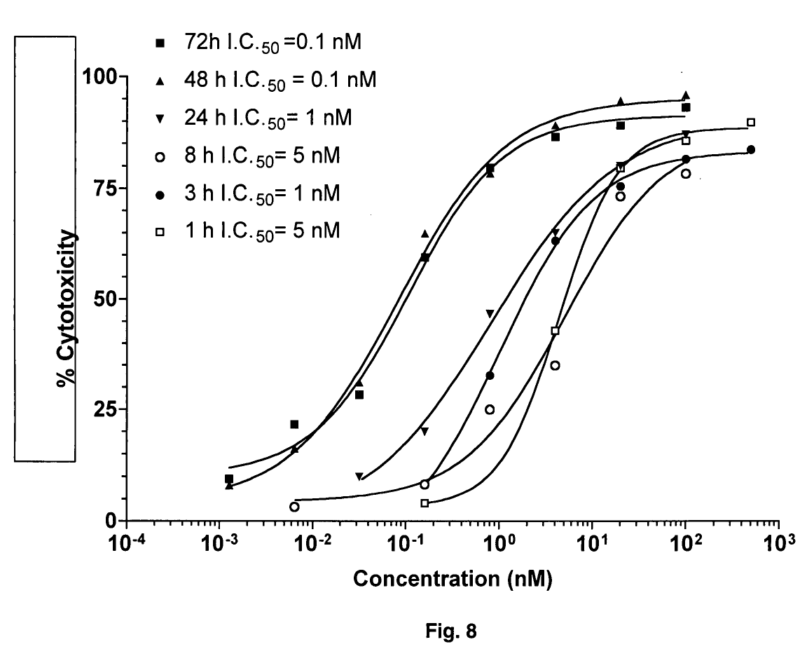

While VEGF₁₂₁/rGel retained cytotoxicity even after a one hour incubation, appreciable cytotoxicity was observed after 24 hours and maximal cytotoxic effect of VEGF₁₂₁/rGel on PAE/KDR cells was observed after 48 hours (FIG. 8). The cytotoxic effect of VEGF₁₂₁/rGel on PAE/FLT-1 cells was also affected as a function of exposure duration (data not shown).

### EXAMPLE 13

Cytotoxic Mechanism of VEGF₁₂₁/rGel

In order to investigate the mechanism of cytotoxicity of VEGF₁₂₁/rGel to PAE/KDR cells, a TUNEL assay was performed for 24, 48 and 72 hours. Log phase PAE/KDR and PAE/FLT-1 cells were diluted to 2000 cells/100 μl. Aliquots (100 μl) were added in 16-well chamber slides (Nalge Nunc International) and incubated overnight at 37° C. with 5% CO₂. Purified VEGF₁₂₁/rGel was diluted in culture media and added at 72, 48 and 24 hour time points at a final concentration of 1 nM (twice the IC₅₀). The cells were then processed and analyzed for TUNEL as described by the manufacturer of the reagent. Positive control cells were incubated with 1 mg/ml DNAse for 10 minutes at 37° C.

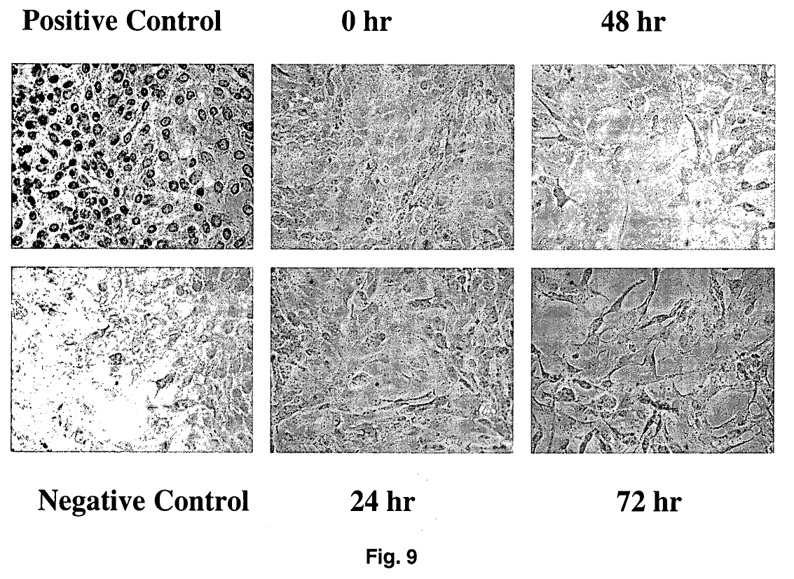

No TUNEL staining was observed with PAE/KDR cells exposed to VEGF₁₂₁/rGel up to 72 hours (FIG. 9). In contrast nuclei of positive control cells showed intense staining, indicating that the mechanism of cytotoxicity of VEGF₁₂₁/rGel is not apoptotic.

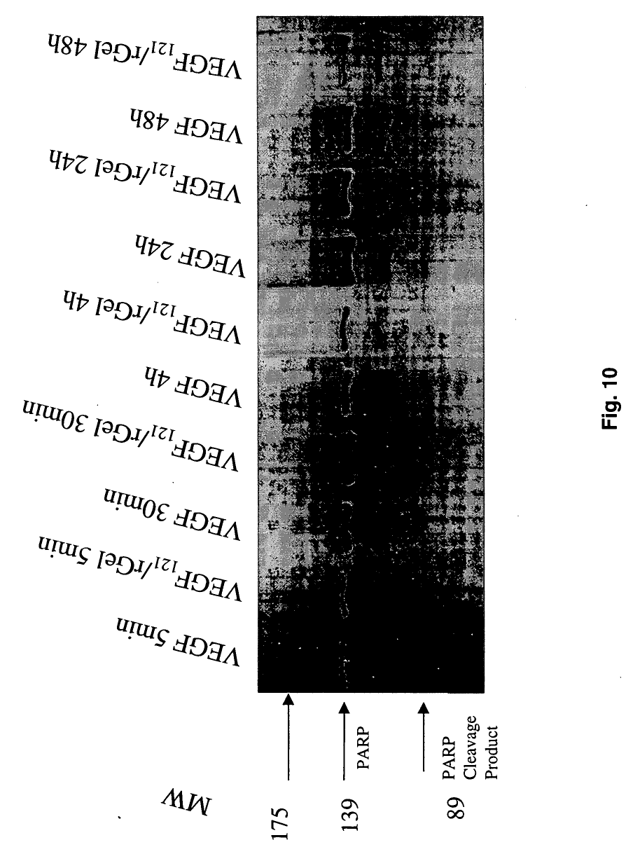

Effects of VEGF₁₂₁/rGel on PARP-mediated apoptosis were investigated by pre-incubating PAE/KDR cells with 100 mM Na₂VO₄ for 5 minutes at 37° C. followed by stimulation with VEGF₁₂₁/rGel or VEGF₁₂₁ for 5 minutes, 30 minutes, 4 h, 24 h and 48 h. Cells were washed and lysed. Cell lysate was analyzed by Western using an anti-PARP antibody. Western blot analysis of these cells showed that VEGF₁₂₁/rGel did not activate PARP-mediated apoptosis (FIG. 10).

### EXAMPLE 14

Inhibition of Tumor Growth in vivo by VEGF₁₂₁/rGel

Human melanoma xenograft model was established as follows. Female nu/nu mice were divided into groups of five mice each. Log-phase A-375M human melanoma cells were injected s.c. (5×10⁶ cells per mouse) into the right flank. After the tumors had become established (˜50 mm³), the mice were injected with VEGF₁₂₁/rGel through a tail vein five times over an 11 day period. The total dose of VEGF₁₂₁/rGel was 17 or 25 mg/kg. Other mice received rGel alone at a dose totaling 10 mg/kg. Mice were killed by cervical dislocation after the 40th day of tumor measurement.

Human prostate cancer xenograft model was established as follows. Male nude mice weighing ˜20 g were divided into groups of five mice each. Log-phase PC-3 human prostate tumor cells were injected s.c. (5×10⁶ cells per mouse) in the right flank. The mice were injected with VEGF₁₂₁/rGel through a tail vein every 2-3 days for 11 days. The total dose of VEGF₁₂₁/rGel was 20 mg/kg. Other mice received rGel alone at a dose totaling 10 mg/kg. Tumor volume was calculated according to the formula: volume=L×W×H, where L=length, W=width, H=height.

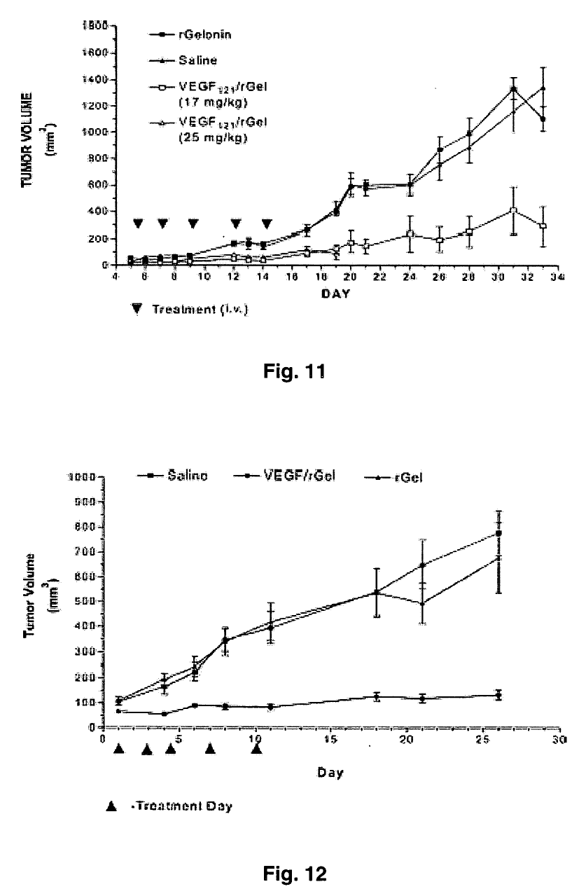

As shown in FIG. 11, saline-treated human melanoma A-375M tumors showed an increase in tumor volume 24-fold (from 50 mm³ to 1200 mm³) over the 30-day observation period. Treatment of the mice with VEGF₁₂₁/rGel strongly retarded tumor growth. At high doses of VEGF₁₂₁/rGel totaling 25 mg/kg, tumor growth was completely prevented, but all mice died from drug toxicity on day 19. At lower doses totaling 17 mg/kg, all mice survived. Tumor growth was completely prevented throughout the 14-day course of treatment, but thereafter, tumor regrowth slowly recurred. Compared with controls, mice treated with VEGF₁₂₁/rGel at doses totaling 17 mg/kg showed a 6-fold decrease in tumor volume (1,200 mm³ vs. 200 mm³).

Human prostatic carcinoma (PC-3) tumors increased 12-fold in volume during the 26-day observation period (FIG. 12). Treatment of the mice with five doses of VEGF₁₂₁/rGel totaling 20 mg/kg virtually abolished tumor growth, even after cessation of treatment. Tumor volume in the treated group only increased from 100 to 200 mm³ over the course of the experiment. Compared with controls, treatment with VEGF₁₂₁/rGel resulted in a 7-fold decrease in tumor volume (1,400 mm³ vs. 200 mm³).

### EXAMPLE 15

Localization of VEGF₁₂₁/rGel to Vascular Endothelium in Prostate Tumor Xenografts

Mice (three mice per group) bearing PC-3 human prostate tumors were injected intravenously with 50 ug of the fusion protein gelonin (2.5 mg/kg) or free gelonin (1 mg/kg). The mean tumor volume per group was 260 mm³. Thirty minutes later, mice were killed, exsanguinated, and all major tissues were snap frozen. Frozen sections were cut and double stained with pan-endothelial marker MECA-32 (5 mg/ml) followed by detection of the localized fusion protein using rabbit anti-gelonin antibody (10 mg/ml). MECA-32 rat IgG was visualized with goat anti-rat IgG conjugated to FITC (red fluorescence). Anti-gelonin antibody was detected with goat anti-rabbit IgG conjugated to Cy-3 (green fluorescence). Colocalization of both markers was indicated by a yellow color. Anti-gelonin antibody had no reactivity with tissue sections from mice injected with saline or VEGF₁₂₁. To determine the percentage of vessels with localized fusion protein, the number of vessels stained with MECA-32 (red), gelonin (green), or both (yellow) were counted at a magnification of ×200 in at least 10 fields per section. Two slides from each mouse were analyzed, and the average percentage of positive vessels was calculated.

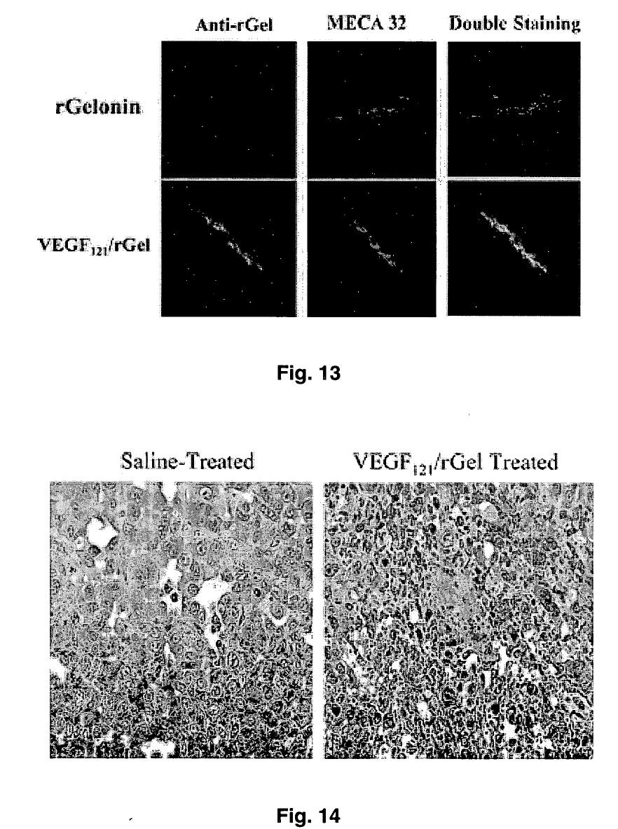

As shown in FIG. 13, VEGF₁₂₁/rGel was detected primarily on vascular endothelium of PC-3 tumors (FIG. 13). On average, 62% of vessels positive for MECA 32 were also positive for VEGF₁₂₁/rGel, as detected by using anti-gelonin antibody. In tumor regions of increased vascularity (“hot spots”), approximately 90% of tumor vessels had bound VEGF₁₂₁/rGel. Vessels in normal organs were unstained, with the exception of the kidney, where weak and diffuse staining was detected in the glomeruli. Free gelonin did not localize to tumor or normal vessels in any of the mice. These results indicate that VEGF₁₂₁/rGel localized specifically to tumor vessels after i.v. injection.

### EXAMPLE 16

Destruction and Thrombosis of Tumor Vessels by VEGF₁₂₁/rGel

Mice bearing s.c. PC-3 tumors were given one i.v. dose of VEGF₁₂₁/rGel (2.5 mg/kg) or saline. The mice were killed 48 h later, and the tumors and various organs were removed. Paraffin sections were prepared and stained with hematoxylin and eosin. The tumors from VEGF₁₂₁/rGel recipients (FIG. 14) displayed damaged vascular endothelium, thrombosis of vessels, and extravasation of RBC components into the tumor interstitium. Normal tissues had un-damaged vasculature. Treatment of mice with saline had no effect on tumor or normal tissues. As assessed by image analysis, necrotic areas of the tumor increased from ˜4% in saline-treated mice to ˜12% after treatment with the fusion construct.

### EXAMPLE 17

Cytotoxicity of VEGF₁₂₁/rGel On MDA-MB-231 Breast Tumor Cells

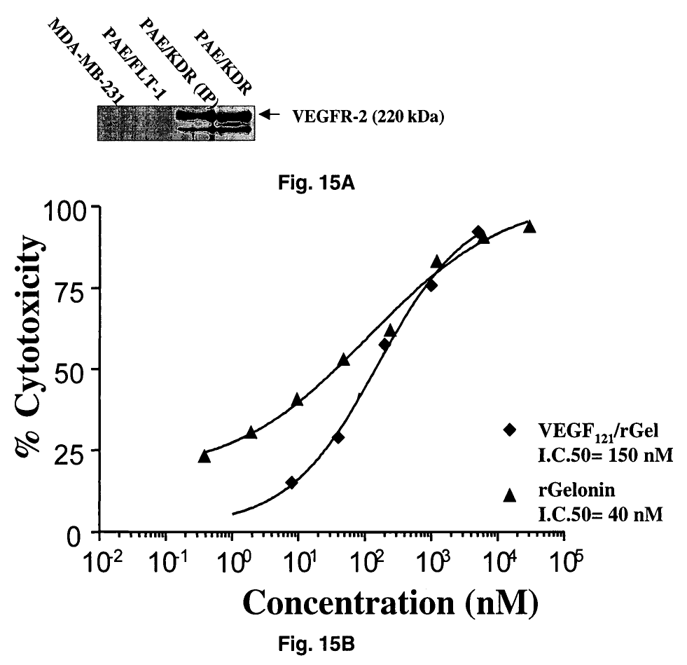

As assessed by Western blot, MDA-MB-231 breast cancer cells do not appear to express VEGFR-1 or VEGFR-2, the receptors which bind VEGF₁₂₁ (FIG. 15A). Cytotoxicity of VEGF₁₂₁/rGel and rGel against log phase MDA-MB-231 cells was determined as follows. Cells were grown in 96-well flat-bottom tissue culture plates. Purified VEGF₁₂₁/rGel and rGel were diluted in culture media and added to the wells in 5-fold serial dilutions. Cells were incubated for 72 hours. The remaining adherent cells were stained with crystal violet (0.5% in 20% methanol) and solubilized with Sorenson's buffer (0.1 M sodium citrate, pH 4.2 in 50% ethanol). Absorbance was measured at 630 nm. As shown in FIG. 15B, the cytotoxicity of VEGF₁₂₁/rGel on MDA-MB-231cells showed an IC₅₀ slightly higher than that observed for recombinant gelonin, indicating that VEGF₁₂₁/rGel does not have a specific target on MBA-MB-231 cells.

### EXAMPLE 18

Localization of VEGF₁₂₁/rGel to Vascular Endothelium in Breast Tumor Xenografts

SCID mice (3 mice per group) bearing orthotopic MDA-MB-231 tumors were intravenously injected with 50 ug of the fusion protein or equivalent amount of free gelonin. The mean tumor volume per group was 260 mm³. Four hours later the mice were sacrificed and exsanguinated. All major organs and tumor were harvested and snap-frozen for preparation of cryosections.

Frozen sections were double stained with a pan-endothelial marker MECA 32 (5 ug/ml) followed by detection of the localized fusion protein using rabbit anti-gelonin antibody (10 ug/ml). MECA 32 rat IgG (provided by Dr. E. Butcher of Stanford University, CA) was visualized by goat anti-rat IgG conjugated to FITC (green fluorescence). Rabbit anti-gelonin antibody was detected by goat anti-rabbit IgG conjugated to Cy-3 (red fluorescence).

Co-localization of both markers was indicated by yellow color. Anti-gelonin antibody had no reactivity with tissues sections derived from mice injected with saline or with VEGF₁₂₁. To determine % of vessels with localized fusion protein, MECA 32 positive, gelonin-positive and vessels with combined color were counted at magnification of ×200 in at least 10 fields per section. Two slides from each mouse were analyzed and percent of positive vessels was averaged.

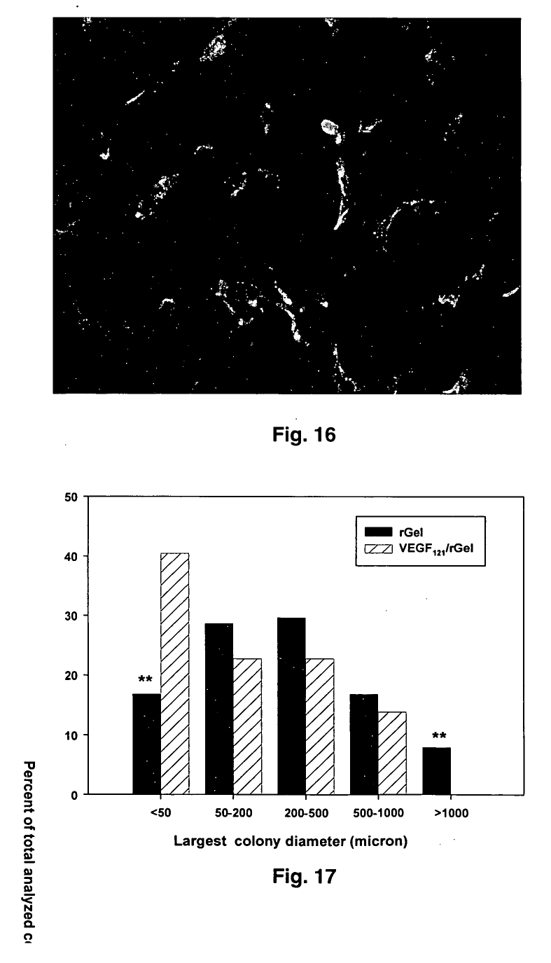

As shown in FIG. 16, VEGF₁₂₁/rGel was primarily detected on endothelium of tumor. In average, sixty percent of vessels positive for MECA 32 were also positive for gelonin in the group of VEGF₁₂₁/rGel-injected mice. In the tumor regions of increased vascularity (hot spots), up to 90% of tumor vessels were labeled by anti-gelonin IgG. Vessels with bound VEGF₁₂₁/rGel were homogeneously distributed within the tumor vasculature. Vessels in normal organs were unstained with the exception of the kidney where weak and diffuse staining was detected in the glomeruli. Free gelonin did not localize to tumor or normal vessels in any of the mice, indicating that only targeted gelonin was able to bind to the tumor endothelium. These results indicate that VEGF₁₂₁/rGel specifically localizes to tumor vessels that demonstrate high density and favorable distribution of VEGF₁₂₁/rGel-binding sites.

### EXAMPLE 19

Metastatic Model of MDA-MB-231 Tumors

The following examples utilize a breast cancer pulmonary metastatic model to establish VEGF₁₂₁/rGel fusion toxin can inhibit metastatic spread and vascularization of metastases.

Human breast carcinoma MDA-MB-231 cells consistently lodge in lungs following intravenous injection into the tail vein of athymic or SCID mice. Micrometastases are first detected 3 to 7 days after injection of 5×10⁵ cells and macroscopic colonies develop in 100% of the injected mice within 4 to 7 weeks. Mortality occurs in all mice within 10-15 weeks. This model of experimental breast cancer metastasis examines the ability of tumor cells to survive in the blood circulation, extravasate through the pulmonary vasculature and establish growing colonies in the lung parenchyma.

Female SCID mice, aged 4-5 weeks, were injected in a tail vein with 0.1 ml of MDA-MB-231 cell suspension (5×10⁵ cells). The mice were randomly separated into two groups (6 mice per group) and were treated with either VEGF₁₂₁/rGel or gelonin alone (100 μg intraperitoneally, 6 times total with an interval of 3 days) starting the 8^{th} day after the injection of cells. Treatment with VEGF₁₂₁/rGel for 2 weeks allow the mice to receive the maximal tolerated accumulative dose of the drug (600 μg per mouse). Prior studies established that VEGF₁₂₁/rGel given at such dose did not cause histopathological changes in normal organs. The accumulative dose of 640-800 μg of total VEGF₁₂₁/rGel fusion protein, given i.p. over period of 4 weeks, did not induce significant toxicity as judged by morphological evaluation of normal organs. Transient loss of weight (˜10%) was observed 24 hours after most of the treatments with complete weight recovery thereafter.

Metastatic colonies were allowed to expand for three weeks after treatment with VEGF₁₂₁/rGel in order to evaluate long-term effect of VEGF₁₂₁/rGel on the size of the colonies, proliferation index of tumor cells and their ability to induce new blood vessel formation. Three weeks after termination of the treatment, the animals were sacrificed and their lungs were removed. One lobe was fixed in Bouin's fixative and the other lobe was snap-frozen. After fixation in Bouin's fixative, the tumor colonies on the lung surface appear white, whereas the normal lung tissue appears brown. The number of tumor colonies on the surface of each lung was counted and the weight of each lung was measured. The values obtained from individual mice in the VEGF₁₂₁/rGel and rGel groups were averaged per group.

### EXAMPLE 20

Effects of VEGF₁₂₁/rGel on the Number, Size and Vascular Density of MDA-MB-231 Pulmonary Metastatic Foci

Frozen samples of lung tissue was cut to produce sections of 6 μm. Blood vessels were visualized by MECA 32 antibody and metastatic lesions were identified by morphology and by 6 w/32 antibody directed against human HLA antigens. Hybridoma producing the mouse monoclonal 6 w/32 antibody was purchased from ATCC. The 6 w/32 antibody was purified from hybridoma supernatant using Protein A resin.

Each section was double stained by MECA 32 and 6 w/32 antibodies to ensure that the analyzed blood vessels are located within a metastatic lesion. Slides were first viewed at low magnification (×2 objective) to determine total number of foci per a cross-section. Six slides derived from individual mice in each group were analyzed and the number was averaged. Images of each colony were taken using digital camera (CoolSnap) at magnifications of ×40 and ×100 and analyzed using Metaview software that allows measurements of smallest and largest diameter, perimeter (μm) and area (mm²).

The vascular endothelial structures identified within a lesion were counted and number of vessels per each lesion was determined and normalized per mm². The mean number of vessels per mm² was calculated per each slide and averaged per VEGF₁₂₁/rGel and rGel groups (6 slides per group). The results are expressed ±SEM. The same method applied to determine the mean number of vessels in non-malignant tissues.

Treatment with VEGF₁₂₁/rGel but not with free gelonin significantly reduced both the number of colonies per lung and the size of the metastatic foci present in lung by 42-58% as shown in FIG. 17 and Table 2.

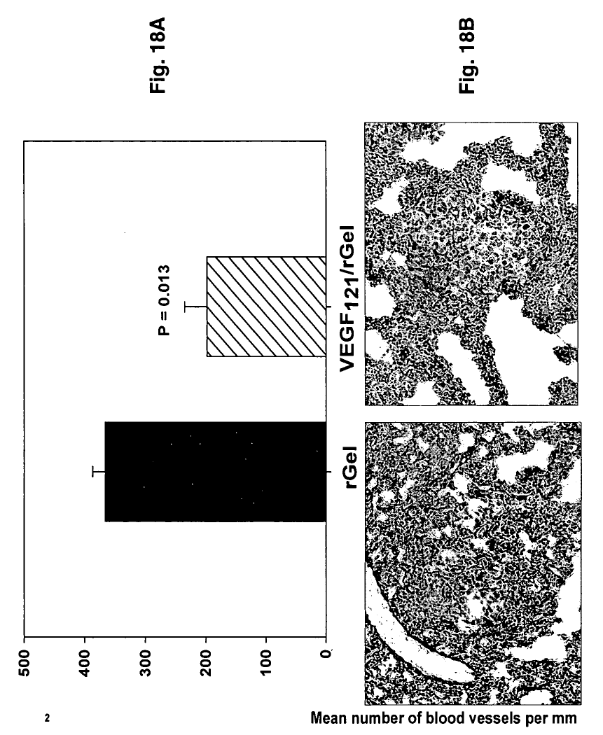

The overall mean vascular density of lung colonies was reduced by 51% compared to the rGel treated controls (Table 3 and FIG. 18). The observed effect, however, was non-uniformly distributed among different tumor colony sizes. The greatest impact on vascularization was observed on mid-size and extremely small tumors (62 and 69% inhibition respectively) while large tumors demonstrated the lease effect (10% inhibition). The majority of lesions in the VEGF₁₂₁/rGel-treated mice (˜70%) were avascular whereas only 40% of lesions from the control group did not have vessels within the metastatic lung foci.

TABLE 2
Effect of VEGF₁₂₁/rGel on Number And Size of Pulmonary
Metastases of MDA-MB-231 Human Breast Carcinoma Cells

| | | | % inhibition vs. | |
|---|---|---|---|---|
| | Treatment^{a} | | rGelonin | |
| Parameter | rGelonin | VEGF₁₂₁/rGel | treatment | P value^{b} |
| No. surface colonies per | 53.3 ± 22 | 22.4 ± 9.2 | 58.0% | 0.03 |
| lung (range)^{c} | (33-80) | (11-41) | | |
| No. intraparenchymal | 22 ± 7.5 | 12.8 ± 5.5 | 42.0% | 0.02 |
| colonies per cross- | (18-28) | (5-18) | | |
| section (range)^{d} | | | | |
| Mean area of colonies(μm)^{e} | 415 ± 10 | 201 ± 37 | 51.9% | 0.01 |
| Mean % of colonies- | 57.3 ± 19 | 25.6 ± 10.5 | 55.4% | 0.01 |
| occupied area per lung | | | | |
| section^{f} | | | | |

^{a}Mice with MDA-MB-231 pulmonary micrometastases were treated i.p. with VEGF₁₂₁/rGel or free gelonin as described.
^{b}P value was calculated using t-Student test.
^{c}Lungs were fixed with Bouin's fixative for 24 hours. Number of surface white colonies was determined for each sample and averaged among 6 mice from VEGF₁₂₁/rGel or rGel control group. Mean number per group ± SEM is shown. Numbers in parentheses represent range of colonies in each group.
^{d}Frozen sections were prepared from metastatic lungs. Sections were stained with 6w/32 antibody recognizing human tumor cells. Number of intraparenchymal colonies identified by brown color was determined for each cross-section and averaged among 6 samples of individual mice from VEGF₁₂₁/rGel or rGel control group. Mean number per group ± SEM is shown. Numbers in parentheses represent range of colonies in each group.
^{e}Area of foci identified by 6w/32 antibody was measured by using Metaview software. Total number of evaluated colonies was 101 and 79 for rGel and VEGF₁₂₁/rGel group, respectively. Six individual slides per each group were analyzed. The mean area of colony in each group ± SEM is shown.
^{f}The sum of all regions occupied by tumor cells and the total area of each lung cross-section was determined and % of metastatic regions from total was calculated. The values obtained from each slide were averaged among 6 samples from VEGF₁₂₁/rGel or rGel control group. The mean % area occupied by metastases from total area per group ± SEM is shown.

TABLE 3
Effect of VEGF₁₂₁/rGel On Vascularity of Pulmonary
Metastases of MDA-MB-231 Human Breast Carcinoma Cells

| | Largest | No. vascularized colonies | | |
|---|---|---|---|---|
| Size of | diameter | from total analyzed (%)^{b} | | % Inhibition vs. |
| colonies^{a} | range (μm) | rGel | VEGF₁₂₁/rGel | radiation treatment |
| Extremely | <50 | 7/24 (29%) | 3/32 (9.3%) | 69 |
| small | | | | |
| Small | 50-200 | 19/48 (39.5%) | 6/24 (25%) | 37 |
| Mid-size | 200-500 | 25/30 (83.3%) | 8/25 (32%) | 62 |
| Large | 500-1000 | 17/17 (100%) | 10/11 (90.0%) | 10 |
| Extremely | >1000 | 8/8 (100%) | N/A | N/A |
| large | | | | |
| No. vascular foci/ | | 76/127 (59.8%) | 27/92 (29.3%) | 51 |
| total analyzed (%)^{c} | | | | |

^{a}Colonies identified on each slide of a metastatic lung were subdivided into 5 groups according to their largest diameter.
^{b}Frozen lung sections from VEGF₁₂₁/rGel or rGel treated mice were stained with MECA 32 antibody. A colony was defined as vascularized if at least one blood vessel branched out from the periphery and reached a center of the lesion. Six slides per each group derived from individual mice were analyzed and data were combined.
^{c}Total number of the analyzed colonies was 127 and 92 for rGel and VEGF₁₂₁/rGel treated groups, respectively. Seventy percent of foci in the VEGF₁₂₁/rGel-treated group were avascular whereas only 40% of lesions from the control group did not have vessels within the metastatic foci.

### EXAMPLE 21

Effect of VEGF₁₂₁/rGel on the Number of Cycling Cells in the MDA-MB-231 Pulmonary Metastatic Foci

Frozen sections of normal mouse organs and metastatic lungs were fixed with acetone for 5 min and rehydrated with PBST for 10 min. All dilutions of antibodies were prepared in PBST containing 0.2% BSA. Primary antibodies were detected by appropriate anti-mouse, anti-rat or anti-rabbit HRP conjugates (Daco, Carpinteria, Calif.). HRP activity was detected by developing with DAB substrate (Research Genetics).

To determine the number of cycling cells, tissue sections were stained with the ki-67 antibody (Abcam, Inc., Cambridge, UK) followed by anti-mouse IgG HRP conjugate. Sections were analyzed at magnification of ×100. Number of cells positive for ki-67 was normalized per mm². The mean number ±SD per VEGF₁₂₁/rGel or control group is presented. The average numbers derived from analysis of each slide were combined per either VEGF₁₂₁/rGel or rGel group and analyzed for statistical differences.

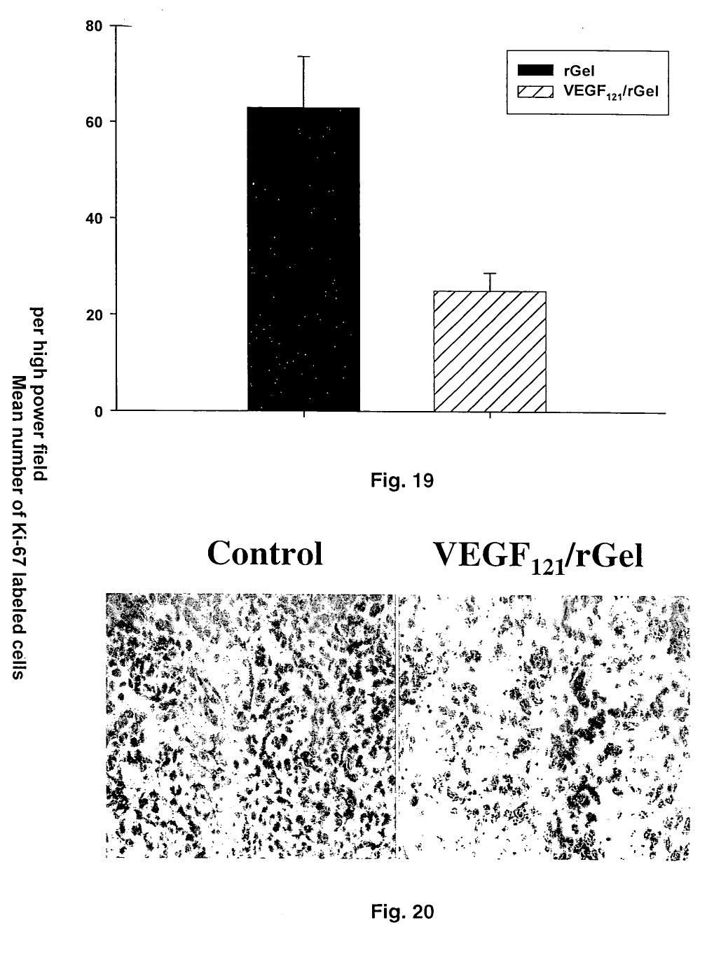

The number of cycling tumor cells in lesions from the VEGF₁₂₁/rGel group was reduced by ˜60% as compared to controls (FIG. 19). The overall mean vascular density of lung colonies was reduced by 51% (Table 3 and FIG. 18). These findings suggest that vascularity of metastases directly affects tumor cell proliferation.

### EXAMPLE 22

Effect of VEGF₁₂₁/rGel on flk-1 Expression in Tumor Vessel Endotheluim of the MDA-MB-231 Pulmonary Metastatic Foci

The expression of VEGF receptor-2 on the vasculature of breast tumors metastatic to lung was assessed using the RAF-1 antibody. Frozen sections of lungs from mice treated with VEGF₁₂₁/rGel or free gelonin were stained with monoclonal rat anti-mouse VEGFR-2 antibody RAFL-1 (10 μg/ml). RAFL-1 antibody was detected by goat anti-rat IgG-HRP. The expression of KDR on the remaining few vessels present in lung metastatic foci demonstrated a significant decline compared to that of lung foci present in control tumors (FIG. 20). This suggests that the VEGF₁₂₁/rGel agent is able to significantly down-regulate the receptor or prevent the outgrowth of highly receptor-positive endothelial cells.

### EXAMPLE 23

Summary of the Biological Properties of VEGF₁₂₁/rGel

VEGF₁₂₁/rGel was found to be selectively toxic to dividing endothelial cells overexpressing the KDR/Flk-1 receptor. Nondividing (confluent) endothelial cells were almost 60-fold more resistant than were dividing cells to the fusion construct and also were more resistant to free gelonin (Table 1). These findings accord with those of previous studies that showed conjugates of vascular endothelial growth factor and diphtheria toxin were highly toxic to log-phase cells but were not toxic to confluent endothelial cells. The greater sensitivity of dividing endothelial cells to VEGF-toxin constructs may be because of differences in intracellular routing or catabolism of the construct as observed with other targeted therapeutic agents.

Cytotoxicity studies demonstrated that expression of the KDR/Flk-1 receptor is needed for VEGF₁₂₁/rGel to be cytotoxic. Cells overexpressing KDR/Flk-1 (>1×10⁵ sites per cell) were highly sensitive to the VEGF₁₂₁/rGel fusion construct, whereas cells expressing fewer than 0.4×10⁵ sites per cell were no more sensitive to the fusion toxin than they were to free gelonin. Again, the requirement to surpass a threshold level of KDR/Flk-1 for cytotoxicity may contribute to the safety of VEGF₁₂₁/rGel. In normal organs, including the kidney glomerulus and pulmonary vascular endothelium, the level of KDR/Flk-1 may be below that needed to cause toxicity. The number of receptors for vascular endothelial growth factor on endothelial cells in the vasculature of normal organs has been reported to be significantly lower than on tumor vasculature. Indeed, one could not detect binding of VEGF₁₂₁/rGel to normal vascular endothelium in organs other than the kidney, where weak binding was observed. Furthermore, no damage to vascular endothelium was observed in normal organs, including the kidney.

Other gelonin-based-targeted therapeutics also have been observed to become toxic to cells only when a certain threshold level of binding is surpassed. In a recent study of immunotoxins directed against the c-erb-2/HER2/neu oncogene product, immunotoxins were not cytotoxic to tumor cells expressing less than about 1×10⁶ HER2/neu sites per cell. The lack of sensitivity of cells having low levels of receptors is presumably because the cells internalize too little of the toxin or traffic it to compartments that do not permit translocation of the toxin to the ribosomal compartment.

Although VEGF/rGel fusion can bind to both the KDR and FLT-1 receptors, only cells expressing KDR were able to internalize the construct thereby delivering the toxin component to the cytoplasmic compartment. It has been suggested that it is the interaction of vascular endothelial growth factor with the KDR receptor but not the FLT-1 receptor that is responsible for the growth proliferative signal on endothelial cells. Other studies suggest that the KDR receptor is primarily responsible for mediating the vascular permeability effects of VEGF-A. Although FLT-1 receptor may modulate signaling of the KDR receptor and impact monocyte response to vascular endothelial growth factor, its role in neovascularization has not been well-defined.

The presence of FLT-1, even at high levels, does not seem to mediate cellular toxicity of the VEGF₁₂₁/rGel fusion toxin. Although VEGF binds to the FLT-1 receptor, the current study has been unable to demonstrate receptor phosphorylation as a result of ligand binding. It has been suggested that receptor phosphorylation may be required for KDR signaling and internalization. If so, the receptor-fusion-toxin complex may not internalize efficiently after binding to FLT-1 for the fusion protein to be routed to an intracellular compartment from which the toxin can escape to the cytosol. The relative contributions of the FLT-1 and KDR receptors to the biological effects of vascular endothelial growth factor examined by using a monoclonal antibody that blocks the interaction of vascular endothelial growth factor with KDR/Flk-1 but not FLT-1 demonstrate that KDR/Flk-1 is the major receptor determining the vascular permeability-inducing and angiogenic effects of vascular endothelial growth factor in tumors.

Another important observation was that the cytotoxic effects of the VEGF₁₂₁/rGel construct on vascular endothelial cells did not involve an apoptotic mechanism. This is in sharp contrast to studies of other toxins such as ricin A chain (RTA) and pseudomonas exotoxin (PE) which demonstrate generation of apoptotic effects that are mediated, at least in part, by caspase activation. It has been suggested that PE toxins may generate cytotoxic effects through both caspase-dependant and protein synthesis inhibitory mechanisms. Despite the sequence homology of ricin A chain and rGel and the known similarities in their mechanism of action, it appears that these two toxins differ in their pro-apoptotic effects. One possible explanation for the observed differences in apoptotic effects between ricin A chain and the rGel toxin could be in the cell types examined. The cells targeted in the current study of rGel are non-transformed endothelial cells while those in the ricin A chain study were tumor cells.

The exposure duration studies for the VEGF₁₂₁/rGel fusion toxin demonstrate that as little as 1 hr exposure to target cells is required to develop a cytotoxic effect 72 hrs later. However, continual exposure for up to 48 hrs was shown to improve the cytotoxic effect by almost 10 fold. Should pharmacokinetic studies demonstrate a relatively short plasma half-life for this agent, this may suggest that optimal therapeutic effect could be achieved by maintaining blood concentrations of drug at therapeutic concentrations for at least 48 hrs. This could be achieved by frequent interval dosing or continuous infusion but may be important in the development of pre-clinical and clinical dosing strategies.

The antitumor effects of the VEGF₁₂₁/rGel fusion construct against both melanoma and human prostate carcinoma xenografts was impressive in magnitude and prolonged. A-375M and PC-3 cells in culture were resistant to the fusion construct in vitro, despite the reported presence of KDR on the melanoma (but not on PC-3) cells. Therefore, the antitumor effects observed in vivo appear not to be caused by direct cytotoxic effects of VEGF₁₂₁/rGel on the tumor cells themselves. The antitumor effect seems to be exerted indirectly on the tumor cells through specific damage to tumor vasculature. The VEGF₁₂₁ fusion toxin localized to tumor blood vessels after i.v. administration. Vascular damage and thrombosis of tumor blood vessels were observed within 48 h of administration of VEGF₁₂₁/rGel to PC-3 mice, consistent with the primary action of the construct being exerted on tumor vascular endothelium.

VEGF₁₂₁/rGel also has an impressive inhibitory effect on tumor metastases. Administration of the VEGF₁₂₁/rGel construct to mice previously injected (i.v.) with the MDA-MB-231 human breast tumor cells dramatically reduced the number of tumor colonies found in lung, their size and their vascularity. In addition, the number of cycling breast tumor cells within lung metastatic foci was found to be reduced by an average of 60%. In addition to the reduced number of blood vessels present in lung metastases of treated mice, the few vessels present had a greatly reduced expression of VEGFR-2. Therefore, VEGF₁₂₁/rGel demonstrated an impressive, long-term impact on the growth and development of breast tumor metastatic foci found in lung.

The salient finding of the effects of VEGF₁₂₁/rGel construct is that this fusion toxin is specifically cytotoxic to cells over-expressing the KDR receptor for VEGF. However, the human breast MDA-MB-231 cancer cells employed for the metastatic studies described above do not express this receptor and, therefore, were not directly affected by this agent. The antitumor effects of VEGF₁₂₁/rGel observed on the MDA-MB-231 metastases thus appear to be solely the result of targeting tumor vasculature.

Neovascularization is a particularly important hallmark of breast tumor growth and metastatic spread. The growth factor VEGF-A and the receptor KDR have both been implicated in highly metastatic breast. It is of interest to note that administration of VEGF₁₂₁/rGel resulted in a 3-fold decrease in the number of Ki-67 labeled (cycling) cells in the metastatic foci present in lung (FIG. 19). Clinical studies have suggested that tumor cell cycling may be an important prognostic marker for disease-free survival in metastatic breast cancer, but that Ki-67 labeling index, tumor microvessel density (MVD) and neovascularization appear to be independently regulated processes (Honkoop et al., 1998; Vartanian and Weidner, 1994). This is the first report of a significant reduction in tumor labeling index produced by a vascular targeting agent.

The vascular-ablative effects of the VEGF₁₂₁/rGel fusion construct alone were able to partially eradicate lung metastases. Although development of small, avascular, metastatic foci within lung tissue was observed, the growth of larger pulmonary metastases was completely inhibited by treatment with the VEGF₁₂₁/rGel fusion toxin. It is conceivable that combination of VEGF₁₂₁/rGel fusion construct with chemotherapeutic agents or with radiotherapeutic agents that directly damage tumor cells themselves may provide for greater therapeutic effect. Studies of several vascular targeting agents in combination with chemotherapeutic agents have already demonstrated a distinct in vivo anti-tumor advantage of this combination modality against experimental tumors in mice (Siemann et al., 2002). Studies by Pedley et al. (2002) have also suggested that combination of vascular targeting and radioimmunotherapy may also present a potent antitumor combination. Finally, studies combining hyperthermia and radiotherapy with vascular targeting agents have demonstrated enhanced activity against mammary carcinoma tumors in mice (Murata et al., 2001).

### EXAMPLE 24

Targeting Osteoclast Precursor Cells By VEGF₁₂₁/rGel

The present example shows that VEGF₁₂₁/rGel strongly inhibits the growth of prostate cancer cells PC-3 placed in the bone micro-environment in mice. Inhibition of tumor growth may not be solely due to targeting by VEGF₁₂₁/rGel of the tumor endothelium, but may also be due to targeting of osteoclast precursor cells. It is hypothesized that VEGF₁₂₁/rGel not only targets the tumor vasculature in the bone microenvironment but also prevents osteoclast maturation.

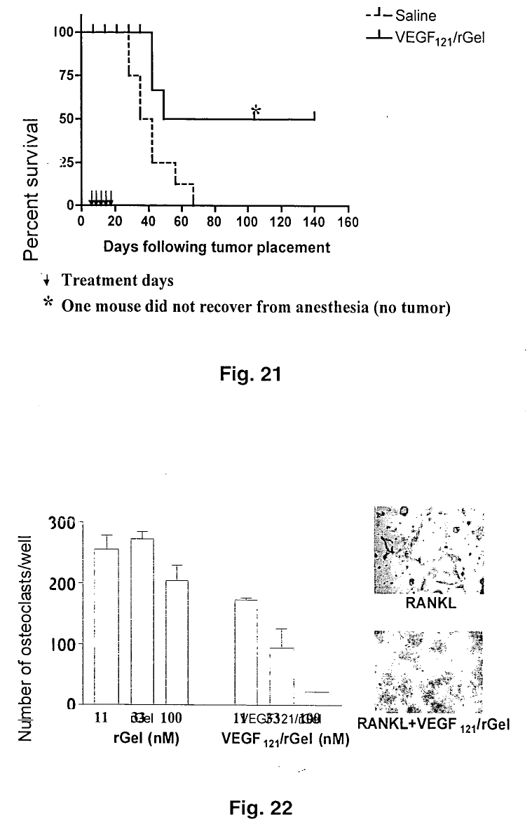

In a prostate cancer bone metastatic model, VEGF₁₂₁/rGel confers significant and impressive survival advantage on mice implanted with PC-3 prostate cancer cells in their femur (FIG. 21). All control mice were sacrificed by day 67 due to large osteolytic lesions or bone lysis. In contrast, 50% of the VEGF₁₂₁/rGel-treated mice survived past day 140 without sign of osteolysis.

To further examine the effect of VEGF₁₂₁/rGel in the bone microenvironment, the effects of VEGF₁₂₁/rGel were examined in mouse myeloid cell line Raw 264.7, an osteoclast precursor cell. Treatment of Raw 264.7 cells with VEGF₁₂₁/rGel inhibited RANKL-mediated osteoclastogenesis (FIG. 22). The observed effect was not mediated by either VEGF₁₂₁ or gelonin alone but was a characteristic unique to the combined fusion protein. Because data presented above indicate that the cytotoxicity of VEGF₁₂₁/rGel on endothelial cells is mediated through Flk-1/KDR and not Flt-1, it is hypothesized that Flk-1/KDR plays an important but as yet unknown role in RANKL-mediated osteoclastogenesis. It is also possible that the biology of VEGF receptors is different in osteoclasts compared to endothelial cells, and VEGF₁₂₁/rGel is able to inhibit formation of osteoclasts via Flt-1.

Thus, VEGF₁₂₁/rGel may be targeting the tumor neovasculature as well as osteoclast precursor cells in vivo. This is significant because VEGF₁₂₁/rGel may inhibit prostate cancer osteoblastic lesions in bone as a result of osteoclastogenesis inhibition.

### EXAMPLE 25

Determine the Effects of VEGF₁₂₁/rGel on Osteoclast Differentiation and Activation in vitro

The effects of VEGF₁₂₁/rGel on differentiation of osteoclast precursors can be examined in two in vitro model systems: (1) mouse osteoclast precursor cells RAW 264.7 that differentiate into mature osteoclasts upon stimulation with RANKL, and (2) bone marrow-derived macrophages that require stimulation with macrophage colony stimulating factor (MCSF) followed by RANKL for differentiation into osteoclasts. In both model systems, cells can be treated with VEGF₁₂₁/rGel, rGel, VEGF₁₂₁, or vehicle simultaneously with RANKL (or MCSF in the case of bone marrow-derived macrophages). Cells are allowed to differentiate for 4-5 days, followed by staining for tartrate-resistant acid phosphatase (TRAP). The dose-dependent effect of each protein can be quantitated by counting the number of TRAP-positive osteoclasts per well. Specificity of VEGF₁₂₁/rGel can be further tested by first treating the cells with increasing doses of VEGF₁₂₁ for one hour, followed by addition of VEGF₁₂₁/rGel or rGel, and monitoring for osteoclast formation.

To test if the effect of VEGF₁₂₁/rGel on osteoclastogenesis has functional significance, the effect of VEGF₁₂₁/rGel on bone resorption can be examined using experimental conditions as outlined above, except that the cells are plated on dentine. Six days after initiating osteoclast differentiation, the dentine will be examined for pits characteristic of bone resorption. Osteoclasts can be identified by TRAP-staining. The presence of bone-resorbing osteoclasts can also be observed in vivo. Relative resorptive area can be quantitated by reflective light microscopy.

It is hypothesized that the inhibition of RANKL-mediated osteoclastogenesis is due to cytotoxic effect of gelonin that enters the cells via the targeted VEGF₁₂₁ receptors. This hypothesis can be tested by performing cytotoxicity assays on Raw 264.7 and bone marrow-derived macrophages with VEGF₁₂₁/rGel and rGel using crystal violet staining as previously described, and determine the IC₅₀ for each drug. A lower IC₅₀ for VEGF₁₂₁/rGel than rGel alone will confirm that VEGF₁₂₁ is important in the specific targeting of these cells. If a cytotoxic effect of VEGF₁₂₁/rGel on Raw 264.7 cells is observed, the mechanism of cell death can be investigated by TUNEL assay and examining caspase-3 cleavage, PARP cleavage as well as changes in cytochrome c, Bax, Bcl and Bcl-xl levels.

### EXAMPLE 26

Cloning of Human Granzyme B Gene and Construction of Granzyme B/VEGF₁₂₁ Fusion Gene

The following examples describe a fusion construct of VEGF₁₂₁ and proapoptotic enzyme granzyme B (GrB) designed for specific delivery to tumor neovasculature. Human granzyme B gene was cloned from human cutaneous T-cell lymphoma (HuT-78) cells and then fused to VEGF₁₂₁ via a short, flexible tether using a PCR-based construction method. The fusion protein was expressed in Escherichia coli and purified by nickel-NTA metal affinity chromatography. The fusion protein GrB/VEGF₁₂₁ was characterized and the biological activities and mechanism were determined.

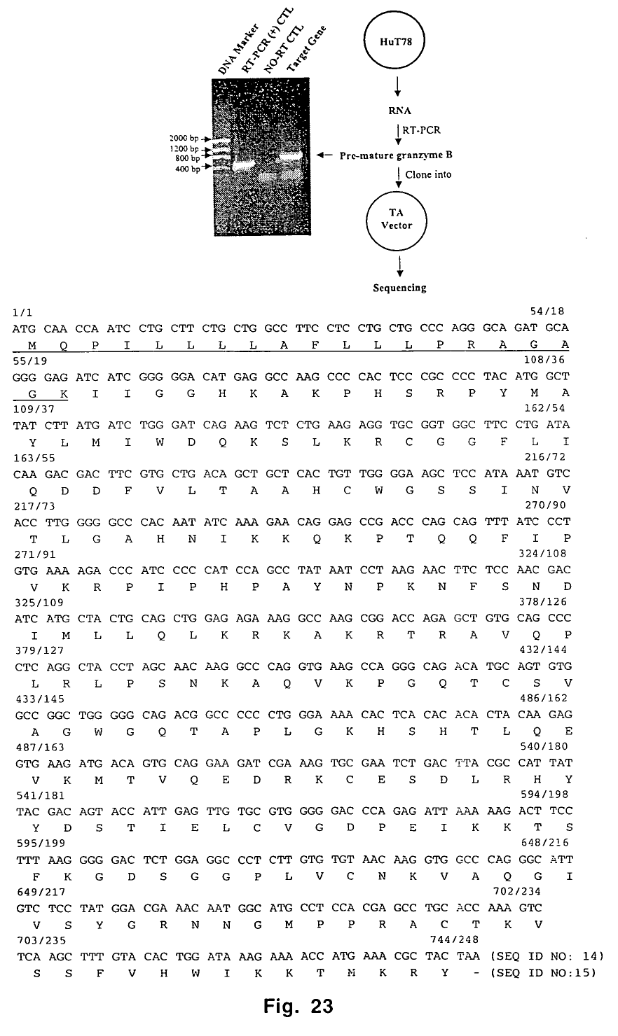

RNA from HuT-78 cells was isolated and target premature human granzyme B cDNA was amplified by reverse transcription-PCR using the following primers: NcoIgb, 5′-GGTGGCGGTGGCTCCATGGAACCAATCCTGCTTCTG-3′ (SEQ ID NO. 5 ) and Cxholgb, 5′-GCCACCGCCTCCCTCGAGCTATTAGTAGCGTTTCATGGT-3′ (SEQ ID NO. 6). The human granzyme B sequence with signal sequence was designated as premature granzyme B (˜800 bp) (FIG. 23). The PCR product was then cloned into the PCR 2.1 TA vector designated as gbTA. The gbTA was transformed into INVaF′ competent cells, and positive clones were screened by PCR. The DNA from positive clones was isolated and then sequenced. The correct clone was designated gbTA-2.

In cytotoxic cells, active granzyme B was generated from a zymogen by dipeptidyl peptidase I-mediated proteolysis, which removes the two residue (Gly-Glu) propeptide and exposes Ile²¹. The amino-terminal Ile-Ile-Gly-Gly sequence of granzyme B is necessary for the mature, active granzyme B.

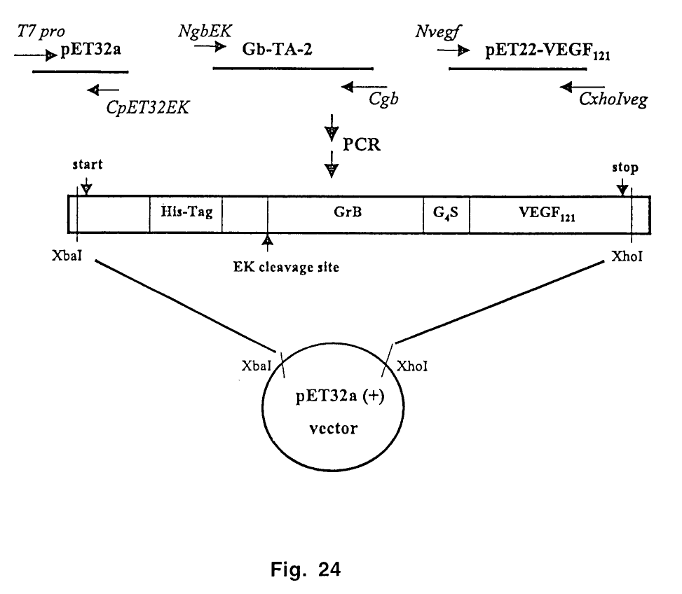

To construct GrB/VEGF₁₂₁ fusion gene, the coding sequence of granzyme B was amplified by PCR from Ile²¹, effectively deleting the signal sequence and Gly-Glu domain. At the same time, a cleavage site for EK (DDDDK, SEQ ID NO. 7) was inserted upstream and adjacent to Ile²¹. Mature granzyme B was attached to the recombinant VEGF₁₂₁ carrier via flexible tether (G₄S). The fused gene fragment was then introduced into the XbaI and XhoI sites of the pET32a(+) to form the expression vector pET32GrB/VEGF₁₂₁ (FIG. 24). This vector contains a T7 promoter for high-level expression followed by a Trx.tag, a His.tag, a thrombin cleavage site, and an EK cleavage site for final removal of the protein purification tag. Once protein tag is removed by rEK digestion, the first residue (isoleucine) of mature granzyme B is exposed, thereby activating the granzyme B moiety of GrB/VEGF₁₂₁ construct.

The GrB/VEGF₁₂₁ fusion gene was constructed by an overlap PCR method. Briefly, granzyme B coding sequence was amplified from gbTA-2 using the following primers: NgbEK, 5′-GGTACCGACGACGACGACAAGATCATCGGGGGACATGAG-3′ (SEQ ID NO. 8) and Cgb, 5′-GGAGCCACCGCCACCGTAGCGTTTCATGGT-3′ (SEQ ID NO. 9). These were designed to delete the signal sequence of premature granzyme B, insert an EK cleavage site at the amino terminus, and add a G4S linker sequence to the carboxyl terminus to serve as a link to the VEGF₁₂₁ gene. VEGF₁₂₁ sequence was amplified from a plasmid pET22-VEGF₁₂₁ (from Dr. Philip Thorpe, University of Texas Southwestern Medical Center, Dallas, Tex.) using the following primers: Nvegf, 5′-GGTGGCGGTGGCTCCGCACCCATGGCAGAA-3′ (SEQ ID NO. 10) and CxhoI veg, 5′-AAGGCTCGTGTCGACCTCGAGTCATTACCGCCTCGGCTTGTC-3′ (SEQ ID NO. 11). To clone the fused genes into pET32a(+) vector with an EK site at the amino terminus of granzyme B, the fragment from pET32a(+) was amplified using the following primers: T7 promoter, 5′-TAATACGACTCACTATAG (SEQ ID NO. 12) and CpET32EK, 5′-CTTGTCGTCGTCGTCGGTACCCAGATCTGG-3′ (SEQ ID NO. 13). The primer has an EK site at carboxyl terminus overlapping with the amino terminus of the fused gene. Using overlap PCR, the fusion genes (EK-GrB/VEGF₁₂₁) were constructed using as primers the T7 promoter and CxhoI veg. Amplified fragments were purified, digested with XbaI and XhoI, and cloned into pET32a(+) vector, designed as pET32GrB/VEGF₁₂₁. A correct clone was chosen for transformation into AD₄₉₄ (DE₃) pLysS-competent cells for further induction and expression.

### EXAMPLE 27

Expression and Purification of Granzyme B/VEGF₁₂₁ Fusion Protein

Bacterial colonies transformed with the constructed plasmid were grown in Luria broth medium (containing 400 mg/ml carbenicillin, 70 mg/ml chloramphenicol, and 15 mg/ml kanamycin) at 37° C. overnight at 240 rpm in a shaking incubator. The cultures were then diluted 1:100 in fresh Luria broth +antibiotics (200 mg/ml ampicillin, 70 mg/ml chloramphenicol, and 15 mg/ml kanamycin) and grown to A_{600 nm}=0.6 at 37° C.; thereafter, isopropyl-1-thio-b-D-galactopyranoside was added to a final concentration of 100 mM and the cells were incubated at 37° C. for 2 h to induce fusion protein expression. The cells were harvested, resuspended in 10 mM Tris (pH 8.0), and stored frozen at −80° C. for later purification.

Thawed, resuspended cells were lysed by addition of lysozyme to a final concentration of 100 mg/ml with agitation for 30 min at 4° C. followed by sonication. Extracts were centrifuged at 186,000×g for 1 h. The supernatant containing only soluble protein was adjusted to 40 mM Tris, 300 mM NaCl, and 5 mM imidazole (pH 8.0) and applied to nickel-NTA agarose resin equilibrated with the same buffer. The nickel-NTA agarose was washed with 300 mM NaCl and 20 mM imidazole and the bound proteins were eluted with 500 mM NaCl and 500 mM imidazole. Absorbance (280 nm) and SDS-PAGE analyses were performed to determine the presence of the polyhistidine-tagged protein, designated as Pro-GrB/VEGF₁₂₁. The eluted Pro-GrB/VEGF₁₂₁ was dialyzed against 20 mM Tris-HCl (pH 7.4) and 150 mM NaCl. The GrB moiety of Pro-GrB/VEGF₁₂₁ was activated by the addition of bovine rEK to remove the polyhistidine tag according to the manufacturer's instructions (1 unit of rEK for cleavage of 50 mg protein incubated at room temperature for 16 h). The rEK was removed by EK capture agarose. The protein solution was then passed through a column containing Q-Sepharose to remove non-rEK-digested construct and nonspecific proteins. The product was analyzed by SDS-PAGE to determine purity, and Bio-Rad protein assay was used to determine protein concentration. Samples were then aliquoted and stored at 4° C.

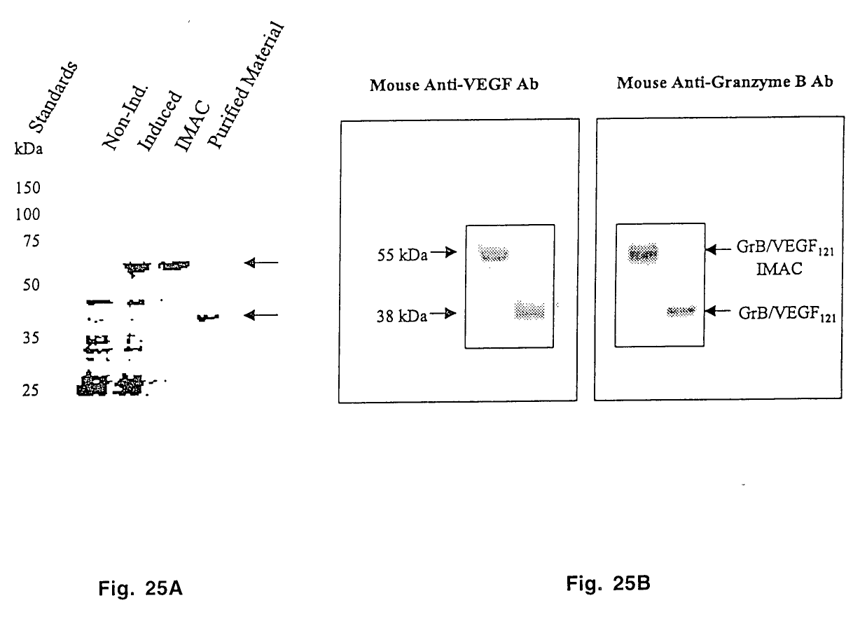

One liter of the culture typically yielded ˜100 mg of the final purified GrB/VEGF₁₂₁ product. SDS-PAGE analysis showed that the final purified GrB/VEGF₁₂, fusion construct migrated under reducing conditions as a band at the expected molecular mass of 38 kDa (FIG. 25A). Specificity of the cleaved fusion protein was confirmed by Western blot using either VEGF121 mouse monoclonal antibody or GrB mouse monoclonal antibody (FIG. 25B).

### EXAMPLE 28

Binding Activity of Granzyme B/VEGF₁₂₁ Fusion Protein

Binding activity of GrB/VEGF₁₂₁ was determined by ELISA. Ninety six-well plates coated with 50,000 cells/well of PAE/FLK-1, PAE/FLT-1, human melanoma A375M or human breast cancer SKBR3-HP cells were blocked by 5% BSA and then treated with purified GrB/VEGF₁₂₁ at various concentrations. After washing, the plates were incubated with either GrB antibody or VEGF₁₂₁ antibody followed by HRP-goat anti-mouse IgG. Then, the substrate 2,2′-azino-bis-3-ethylbenzthiazoline-6-sulfonic acid (ABTS) solution with 1 ml/ml of 30% H₂O₂ was added to the wells. Absorbance at 405 nm was measured after 30 min.

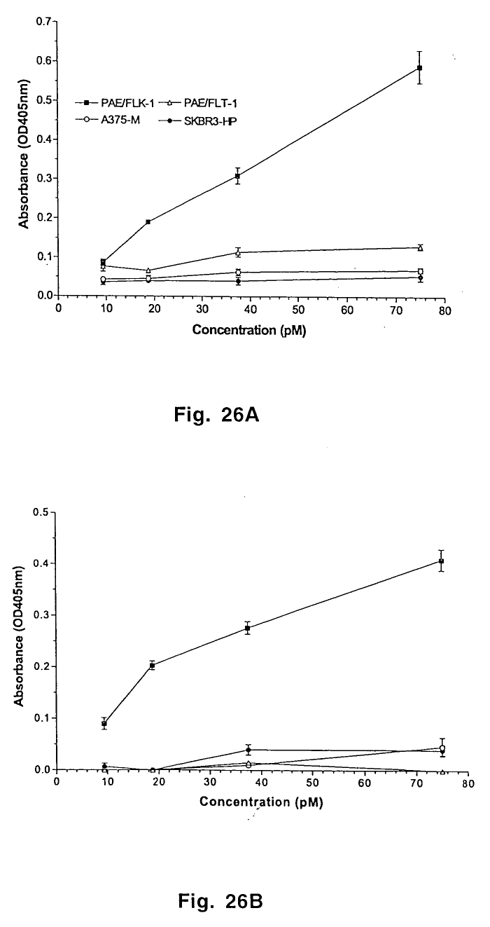

GrB/VEGF₁₂₁ specifically bound to PAE/FLK-1 cells. However, the protein did not bind to PAE/FLT-1 cells or to melanoma A375M or human breast cancer SKBR3-HP cells, as detected by either an anti-GrB mouse monoclonal antibody (FIG. 26A) or an anti-VEGF₁₂₁ mouse monoclonal antibody (FIG. 26B).

### EXAMPLE 29

Internalization of Granzyme B/VEGF₁₂₁ Fusion Protein Assessed by Immunofluorescence Microscopy

Cells were plated in 16-well chamber slides (Nunc, Nalge Nunc International, Naperville, Ill.) at 1×10⁴ cells/well and incubated overnight at 37° C. in a 5% CO₂ air atmosphere. Cells were treated with 100 nM of GrB/VEGF₁₂₁ for 4 h and then washed briefly with PBS. The cell surface was stripped by incubation with glycine buffer (500 mM NaCl, 0.1 M glycine [pH 2.5]) and neutralized for 2 min with 0.5 M Tris (pH 7.4) followed by wash with PBS. Cells were fixed in 3.7% formaldehyde for 15 min at room temperature, permeabilized for 10 min in PBS containing 0.2% Triton X-100 and washed thrice with PBS. Samples were incubated with 3% BSA for 1 h at room temperature to block nonspecific binding sites before incubating with anti-GrB mouse monoclonal antibody (1:100 dilution) at room temperature for 1 h followed by incubation with FITC-coupled anti-mouse IgG (1:100 dilution) at room temperature for I h. The walls and gaskets of the chamber slide were then removed carefully. After air drying, the slide was mounted and analyzed under a Nikon Eclipse TS-100 fluorescence microscope. Photographs were taken with a scope-mounted camera.

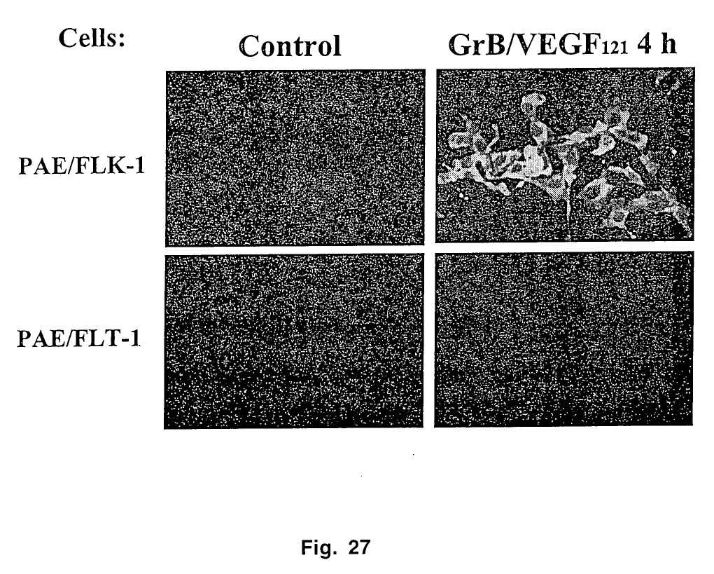

Immunofluorescent staining clearly showed that the GrB moiety of GrB/VEGF₁₂₁ was delivered into the cytosol of PAE/FLK-1 but not into that of PAE/FLT-1 cells after treatment with GrB/VEGF₁₂₁ for 4 h (FIG. 27). Analysis of PAE/FLK-1 cells treated for 24 and 48 h demonstrated no further increase in immunofluorescent staining over that observed at 4 h.

### EXAMPLE 30

Cytotoxicity of Granzyme B/VEGF₁₂₁ Fusion Protein

The cytotoxicity of GrB/VEGF₁₂₁ was assessed against log-phase PAE/FLK-1 and PAE/FLT-1 cells in culture. PAE cells in Ham's F-12 medium with 10% fetal bovine serum were plated into 96-well plates at a density of 2.5×10³ cells/well and allowed to adhere for 24 h at 37° C. in 5% CO₂. After 24 h, the medium was replaced with medium containing different concentrations of GrB/VEGF₁₂₁ or VEGF₁₂₁/rGel. After 72 h, the effect of GrB/VEGF₁₂₁ or VEGF₁₂₁/rGel on the growth of cells in culture was determined using 2,3-bis[2-methoxy-4-nitro-5-sulfophenyl]-2H-tetrazolium-5-carboxanilide inner salt (XTT). Plates were read on a microplate ELISA reader at 540 nm.

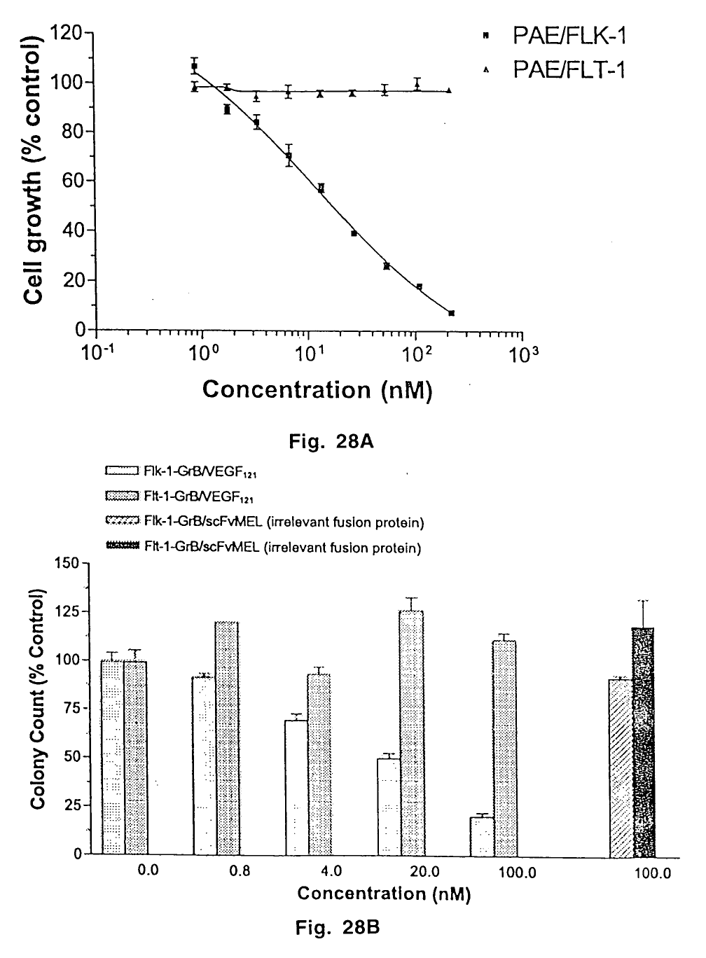

An IC₅₀ effect was found at a concentration of ˜10 nM on PAE/FLK-1 cells. However, no cytotoxic effects were found on PAE/FLT-1 cells at doses up to 200 nM (FIG. 28A). By comparison, the cytotoxic effects of another fusion toxin, VEGF₁₂₁/rGel, were relatively greater (on a molar basis) against target cells in culture and demonstrated specific cytotoxicity against PAE/FLK-1 cells at an IC₅₀ of ˜1 nM.

The growth inhibitory effects of GrB/VEGF₁₂₁ on the proliferation of PAE cells were evaluated by clonogenic assay. Briefly, 5×10⁵ PAE cells/ml were incubated at 37° C. and 5% CO₂ for 72 h with different concentrations of either GrB/VEGF₁₂₁ or 100 nM of irrelevant fusion protein GrB/scFvMEL. Cells were then washed with PBS, trypsinized, counted by hemacytometer, and diluted serially. The serial cell suspensions were then plated in triplicate and cultured in six-well plates for 5-7 days. Cells were stained with crystal violet and colonies consisting of >20 cells were counted using an inverted light microscope. Growth inhibition was defined as the percentage of cell growth/number of colonies in treated samples in relation to that in the nontreated control sample.

In the clonogenic assay (FIG. 28B), the concentration of GrB/VEGF₁₂₁ which suppressed cell colony growth by 50% (IC₅₀) was determined to be ˜20 nM on PAE/FLK-cells. In contrast, there was no effect on colony growth of PAE/FLT-1 cells at concentrations of GrB/VEGF₁₂₁ up to 100 nM. There was also no effect of irrelevant fusion protein GrB/scFvMEL targeting human melanoma cells on colony growth of PAE cells at concentrations of 100 nM.

### EXAMPLE 31

In Situ Cell Death Detection (TUNEL Assay)

Cleavage of genomic DNA during apoptosis may yield double-stranded, low molecular mass DNA fragments as well as single-strand breaks (nicks) in high molecular mass DNA. The DNA strand breaks can be identified by labeling free 3′ hydroxyl termini with modified nucleotides in an enzymatic reaction. Cells (1×10⁴ cells/well) were treated with GrB/VEGF₁₂₁ at the IC₅₀ concentration for different times (24 and 48 h) and washed briefly with PBS. Cells were fixed with 3.7% formaldehyde at room temperature for 20 min, rinsed with PBS, permeablized with 0.1% Triton X-100, 0.1% sodium citrate on ice for 2 min, and washed with PBS twice. Cells were incubated with TUNEL reaction mixture at 37° C. for 60 min followed by incubation with Converter-AP at 37° C. for 30 min and finally treated with Fast Red substrate solution at room temperature for 10 min. After the final wash step, the slides were mounted and analyzed for nucleus staining of apoptotic cells under a light microscope with 400× magnification.

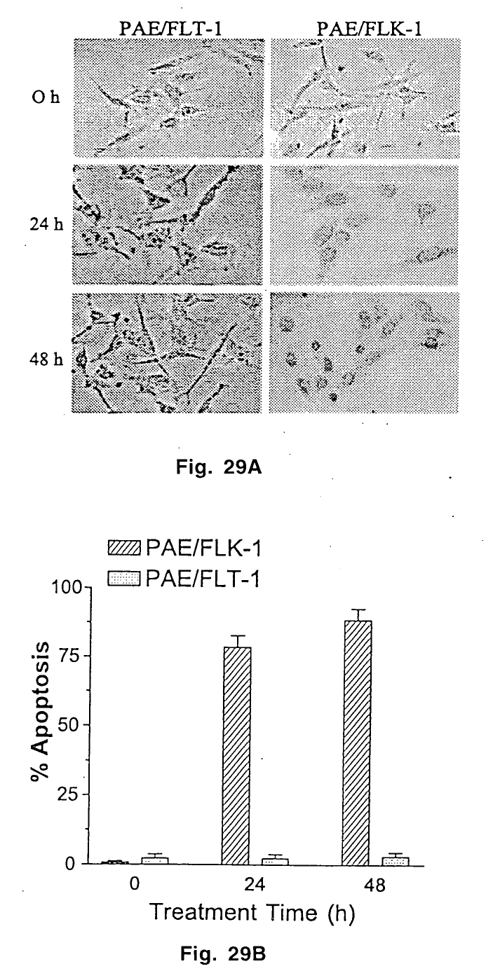

TUNEL assay produced positive results on GrB/VEGF₁₂₁-treated PAE/FLK-1 cells at 24 h (75%) and 48 h (85%) but not on GrB/VEGF₁₂₁-treated PAE/FLT-1 cells (10%) (FIG. 29), indicating that GrB/VEGF₁₂₁ induced apoptosis in PAE/FLK-1 cells.

### EXAMPLE 32

Cytochrome c Release Assay and Bax Translocation

PAE cells (5×10⁷) were treated with GrB/VEGF₁₂₁ at concentrations of 0.1 and 20 nM for 24 h. After cells were washed with 10 ml of ice-cold PBS, they were resuspended with 0.5 ml of 1× cytosol extraction buffer mix containing DTT and protease inhibitors and incubated on ice for 10 min. Cells were homogenized in an ice-cold glass homogenizer. The homogenate was centrifuged at 700×g for 10 min at 4° C. The supernatant was transferred to a fresh 1.5 ml tube and centrifuged at 10,000×g for 30 min at 4° C. The supernatant was collected and labeled as cytosolic fraction. The pellet was resuspended in 0.1 ml mitochondrial extraction buffer mix containing DTT and protease inhibitors, vortexed for 10 s, and saved as mitochondrial fraction. Protein concentrations were determined by using Bio-Rad Bradford protein assay. Aliquots of 30 mg from each cytosolic and mitochondrial fraction isolated from non-treated and treated cells were loaded on a 15% SDS-PAGE. Standard Western blot procedure was performed, and the blot was probed with mouse anti-cytochrome c antibody (1 mg/ml) or mouse anti-Bax antibody (1 mg/ml).

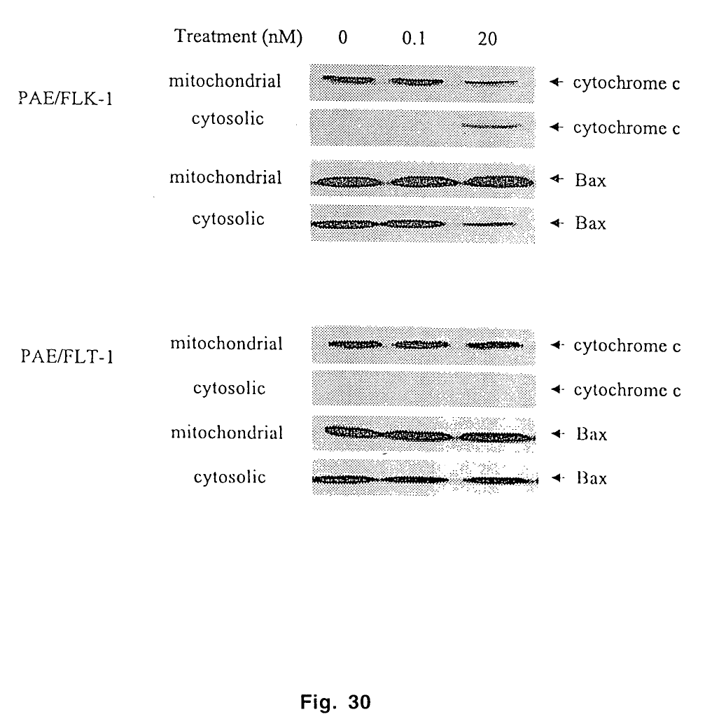

Western blot studies demonstrated that cytochrome c was released from mitochondria into the cytosol after treating PAE/FLK-1 cells with 20 nM GrB/VEGF₁₂₁, but this effect was not observed on PAE/FLT-1 cells (FIG. 30). Bax was found to be normally present in both cytosol and mitochondria of untreated PAE cells. However, when PAE/FLK-1 cells were treated with 20 nM of GrB/VEGF₁₂₁, Bax levels decreased in cytosol and increased in mitochondria. This effect was not observed on PAE/FLT-1 cells (FIG. 30).

### EXAMPLE 33

Granzyme B/VEGF₁₂₁ Induces DNA Laddering

PAE cells were plated onto six-well plates (2×10⁵ cells/well). Twenty-four hours later, cells were shifted to fresh culture medium containing 20 nM of GrB/VEGF₁₂₁ (1.5 ml/well). After 24 h of incubation at 37° C., DNA was extracted and purified with DNA ladder kit (Roche) and fractionated on 1.5% agarose gels.

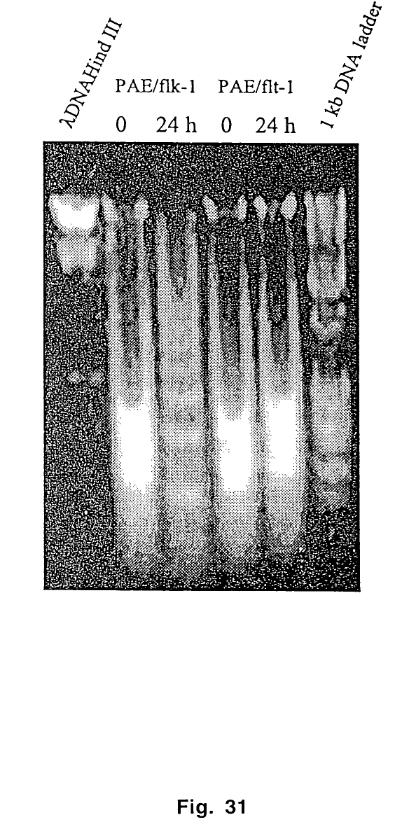

DNA laddering indicative of apoptosis was observed after a 24-h exposure with GrB/VEGF₁₂₁ on PAE/FLK-1 cells. As expected, there was no DNA laddering detected on PAE/FLT-1 cells after treatment with the fusion construct (FIG. 31).

### EXAMPLE 34

Granzyme B/VEGF₁₂₁ Activates Caspases on Porcine Aortic Endothelial Cells

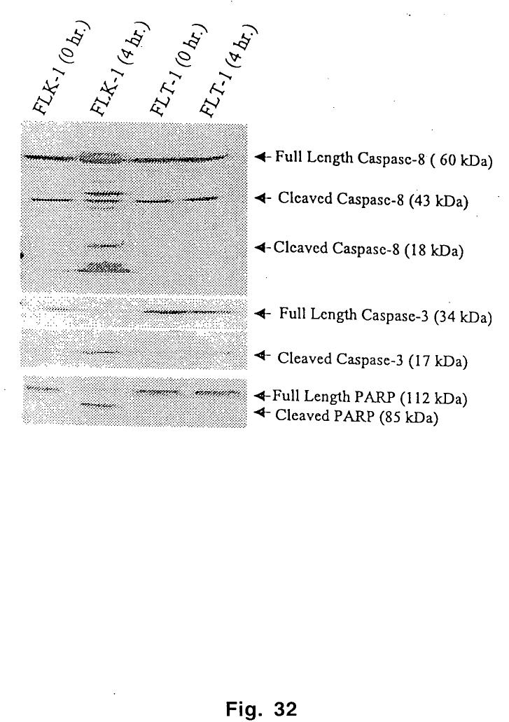

PAE cells were treated with GrB/VEGF₁₂₁, total cell lysates were loaded onto 12% SDS-PAGE, and standard Western blotting was performed. The results showed that treatment with GrB/VEGF₁₂₁ cleaved caspase-8, caspase-3, and PARP on PAE/FLK-1 cells but not on PAE/FLT-1 cells (FIG. 32). These data indicate that the GrB/VEGF₁₂₁ construct activated caspases involved in the apoptosis pathway.

The following references were cited herein:

Brekken, et al., Cancer Res. 58:1952-1959 (1998).

Honkoop et al., Br. J. Cancer 77:621-26 (1998).

Murata et al., Int. J. Radiat. Oncol. Biol. Phys. 51:1018-24 (2001).

Pedley et al., Int. J. Radiat. Oncol. Biol. Phys. 54:1524-31 (2002).

Siemann et al., Int. J. Cancer 99:1-6 (2002).

Vartanian and Weidner, Am. J. Pathol. 144:1188-94 (1994).

Veenendaal et al., Proc Natl Acad Sci USA 99:7866-71 (2002).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A conjugate comprising the 121-amino acid isoform of vascular endothelial growth factor (VEGF₁₂₁) and a cytotoxic molecule.

2. The conjugate of claim 1, wherein said conjugate is a fusion protein of VEGF₁₂₁ and said cytotoxic molecule.

3. The conjugate of claim 1, wherein said cytotoxic molecule is a toxin or a signal transduction protein capable of generating apoptotic signals.

4. The conjugate of claim 3, wherein said toxin is gelonin.

5. The conjugate of claim 3, wherein said signal transduction protein capable of generating apoptotic signals is granzyme B or Bax.

6. The conjugate of claim 1, wherein said VEGF₁₂₁ and said cytotoxic molecule are linked by a linker selected from the group consisting of G₄S, (G₄S)2, 218 linker, (G₄S)3, enzymatically cleavable linker and pH cleavable linker.

7. A pharmaceutical composition comprising the conjugate of claim 1.

8. A method of killing a cell expressing type 2 vascular endothelial growth factor receptor (kinase domain receptor/Flk-1 receptor), comprising the steps of:
contacting said cell with a pharmacologically effective amount of a conjugate comprising the 121-amino acid isoform of vascular endothelial growth factor (VEGF₁₂₁) and a cytotoxic molecule.

9. The method of claim 8, wherein said cytotoxic molecule is a toxin or a signal transduction protein capable of generating apoptotic signals.

10. The method of claim 8, wherein said conjugate is a fusion protein comprising VEGF₁₂₁ and gelonin or a fusion protein comprising VEGF₁₂₁ and granzyme B or Bax.

11. The method of claim 8, wherein said VEGF₁₂₁ and said cytotoxic molecule are linked by a linker selected from the group consisting of G₄S, (G₄S)2, 218 linker, (G₄S)3, enzymatically cleavable linker and pH cleavable linker.

12. The method of claim 8, wherein said conjugate is cytotoxic to cells expressing more than 2000 type 2 VEGF receptors per cell.

13. A method of inhibiting tumor growth or inhibiting metastatic spread and vascularization of metastases in a subject, comprising the step of administering to said subject a biologically effective amount of a conjugate capable of exerting a cytotoxic effect on the tumor vasculature, said conjugate comprises the 121-amino acid isoform of vascular endothelial growth factor (VEGF₁₂₁) and a cytotoxic molecule, wherein said VEGF₁₂₁ binds to both VEGF receptor type 1 (Flt-1) and VEGF receptor type 2 (kinase domain receptor/Flk-1) but is only internalized by cells expressing VEGF receptor type 2.

14. The method of claim 13, wherein said cytotoxic molecule is a toxin or a signal transduction protein capable of generating apoptotic signals.

15. The method of claim 13, wherein said conjugate is a fusion protein comprising VEGF₁₂₁ and gelonin or a fusion protein comprising VEGF₁₂₁ and granzyme B or Bax.

16. The method of claim 13, wherein said VEGF₁₂₁ and said cytotoxic molecule are linked by a linker selected from the group consisting of G₄S, (G₄S)3, (G₄S)2, 218 linker, enzymatically cleavable linker and pH cleavable linker.

17. The method of claim 13, wherein said conjugate is cytotoxic to cells expressing more than 2000 type 2 VEGF receptors per cell.

18. The method of claim 13, further comprises treatment with chemotherapeutic agents or radiotherapeutic agents.

19. A method of inhibiting osteoclastogenesis or treating osteoporosis in a subject, comprising the step of administering to said subject a biologically effective amount of a conjugate comprising the 121-amino acid isoform of vascular endothelial growth factor (VEGF₁₂₁) and a cytotoxic molecule.

20. The method of claim 19, wherein said cytotoxic molecule is a toxin or a signal transduction protein capable of generating apoptotic signals.

21. The method of claim 19, wherein said conjugate is a fusion protein comprising VEGF₁₂₁ and gelonin or a fusion protein comprising VEGF₁₂₁ and granzyme B or Bax.

22. The method of claim 19, wherein said VEGF₁₂₁ and said cytotoxic molecule are linked by a linker selected from the group consisting of G₄S, (G₄S)2, 218 linker, (G₄S)3, enzymatically cleavable linker and pH cleavable linker.
